(19) United States
(12) Patent Application Publication
(10) Pub. No.: US 2013/0217147 A1
(43) Pub. Date: Aug. 22, 2013

(54) DETECTION OF COMPLEXES OF TAU AND AMYLOID

(71) Applicants: Winton G. Gibbons, Winnetka, IL (US); Thomas L. Holzman, Libertyville, IL (US); Lei Chang, Westmont, IL (US); Claude G. Lerner, Highland Park, IL (US)

(72) Inventors: Winton G. Gibbons, Winnetka, IL (US); Thomas L. Holzman, Libertyville, IL (US); Lei Chang, Westmont, IL (US); Claude G. Lerner, Highland Park, IL (US)

(73) Assignee: Nanosphere, Inc., Northbrook, IL (US)

(21) Appl. No.: 13/718,682

(22) Filed: Dec. 18, 2012

Related U.S. Application Data
(63) Continuation of application No. 13/442,671, filed on Apr. 9, 2012, now abandoned.
Application No. 13/442,671 is a continuation of application No. 13/203,171, now abandoned.
(60) Provisional application No. 61/155,151, filed on Feb. 24, 2009.
(60) Provisional application No. 61/155,154, filed on Feb. 24, 2009.
(60) Provisional application No. 61/156,272, filed on Feb. 27, 2009.

(51) Int. Cl. G01N 33/68

(52) U.S. Cl. CPC G01N 33/6896
USPC 436/501

(57) ABSTRACT

The invention relates to methods for detecting complexes of Tau, Tau variants, including phosphorylated variants, and amyloid containing molecules, as well as autoantibodies to those complexes or components of those complexes, in physiological fluid samples.

### CROSS-REFERENCE TO RELATED APPLICATIONS

This application is a Continuation of and claims the benefit of priority under 35 U.S.C. §120 to U.S. patent application Ser. No. 13/442,671, filed on Apr. 9, 2012, which is a Continuation of U.S. application Ser. No. 13/203,171, filed Aug. 24, 2011, which is a U.S. National Stage Filing Under 35 U.S.C. 371 from International Patent Application Serial No. PCT/US2010/025231, filed on Feb. 24, 2010, which claims the benefit of the filing date of U.S. application Ser. No. 61/155,151, filed on Feb. 24, 2009, U.S. application Ser. No. 61/155,154, filed on Feb. 24, 2009, and U.S. application Ser. No. 61/156,272, filed on Feb. 27, 2009, the disclosures of which are incorporated by reference herein.

### TECHNICAL FIELD

The present technology relates generally to diagnostic and prognostic methods for neurological diseases such as mild cognitive impairment (MCI) and Alzheimer's disease. In particular, the present disclosure relates to methods for detecting complexes of Tau, Tau variants, including phosphorylated variants, and amyloid containing molecules, as well as autoantibodies to those complexes or components of those complexes, in physiological fluid samples, which complexes are a marker for disorders including Alzheimer's disease, as well as other neurological diseases such as mild cognitive impairment (MLD).

### BACKGROUND

The pathological hallmarks of Alzheimer's disease (AD) are amyloid plaques, neurofibrillary tangles, synaptic degeneration and neuronal loss (Price et al., Annu. Rev. Neurosci., 21:479 (1998).). Amyloid plaques are composed of amyloid-beta (Aβ) 42 and 40 peptides derived from the proteolytic cleavage of amyloid precursor protein (APP) by β-site APP cleavage enzyme 1 (BACE1) (Sinha et al., Nature, 402:537 (1999); Vassar et al., Science, 286:735 (1999)) and the γ-secretase (De Strooper, Neuron., 38:9 (2003). The endosome and the endocytic pathways have been proposed as possible sites for the β and γ cleavage sites of APP (Small et al., Neuron., 52:15 (2006)), and the resulting Aβ peptides are secreted by both neuronal and non-neuoronal cells (Selkoe, J. Clin. Invest., 110:1375 (2002); Selkoe, Science, 275:630 (1997)). Recently, soluble forms of Aβ have been implicated in neurotoxicity (Lambert et al., Proc. Natl. Acad. Sci. USA, 95:6448 (1998); Walsh et al., Nature, 416:535 (2002)), and may correlate better with cognition than amyloid plaque burden (Lue et al., Am. J. Pathol., 155:853 (1999); McLean et al., Ann. Neurol., 46:860 (1999)).

The clinical manifestations of AD, i.e., cognitive decline and neuro-behavioral changes, are preceded by a long preclinical stage characterized by the silent development of neuropathological lesions (Crystal et al., Neurology, 38:1682 (1988); Katzman et al., Ann. Neurol., 23:138 (1988); Price et al., Ann. Neurol., 45:358 (1999); Schmitt et al., Neurology, 55:370 (2000); Morris et al., J. Mol. Neurosci., 17:101 (2001)). These preclinical and early stages of AD represent the ideal time to treat the disease (Neugroschl, Am. J. Geriatr. Psychiatry, 10:660 (2002)).

As Aβ is considered to play an early and pivotal role in AD pathogenesis (Hardy et al., Science, 297:353 (2002)), it may be a useful tool in diagnosing AD in the preclinical/early stages, as well as for monitoring potential Aβ modifying therapies (Galasko, J. Alzheimers Dis., 8:339 (2005)). While human CSF Aβ levels have mostly shown reduction with disease progression (Jensen et al., Ann. Neurol., 45:504 (1999)), much of the data on plasma Aβ levels have been equivocal (Irizarry et al., J. Neuropathol. Exp. Neurol., 56:965 (1997)).

Aβ, and in particular Aβ₁₋₄₂, has been studied frequently as a biomarker for AD. CSF concentrations of Aβ₁₋₄₂ are reduced by 40% to 50%, whereas concentrations of Aβ₁₋₄₀ or “Aβₜₒₜₐₗ” (using an ELISA that does not distinguish C-terminal length) are similar to those of age-matched controls. CSF Aβ₁₋₄₂ does correlate to an extent with dementia severity; however, in most studies concentrations are stable over intervals as long as 12 months (Andreasen et al., Arch. Neurol., 56:673 (1999)).

Plasma concentrations of Aβ₁₋₄₂ do not correlate with those in CSF (Mehta et al., Neurosci. Lett., 304:102 (2001). Longitudinal studies have not shown a consistent change in plasma Aβ over time in AD patients (Mayeux et al., Neurology, 61:1185 (2003)), and cross-sectional differences between AD patients and controls that would allow plasma Aβ concentrations to be used as a diagnostic measure have not been identified.

Cerebrospinal fluid tau has also been studied as a potential biomarker in AD (Blennow, Neurorx, 1:213 (2004)). Elevations of 2- to 3-fold of CSF total tau (T-tau) levels in patients with AD have been demonstrated in cross-sectional studies. In longitudinal studies, weak correlations are present with changes in cognitive scores, and CSF T-tau levels remain stably elevated in AD over time intervals of 12 months or longer. Tau may be phosphorylated at various sites, and forms of CSF tau reflecting specific sites of phosphorylation (P-tau 181, 199, 231, 235, 396, and 404) have been studied.

Three species of p-tau (p-thr231, p-ser199, and p-thr181) have been examined in detail in cross-sectional studies (Hampel et al., Arch. Gen. Psychiatry, 61:95 (2004); Ishiguro et al., Neurosci. Lett., 270:91 (1999); Vanmechelen et al., Neurosci. Lett., 285:49 (2000); Zetterberg et al., Neurosci. Lett., 352:67 (2003)). All three species are elevated in the CSF of patients with AD, and concentrations of all three species appear to be linearly related. When assessed as diagnostic measures, these three measures have similar sensitivity, although p-thr231 may have somewhat greater specificity for AD versus other forms of dementia (Hampel et al., 2004). Interestingly, p-thr231 tau, as well as other forms, is elevated in MC1 patients compared with control subjects, but longitudinal studies of AD patients show a progressive decline in concentration with disease progression (Hampel et al., Ann. Neurol., 49:545 (2001)).

### SUMMARY OF THE INVENTION

The invention provides a method to detect complexes of Tau and Aβ (Abeta) containing molecules in physiological fluid of a mammal or other test subject at risk of or suspected of having neurological disorders including but not limited to MLD and Alzheimer's disease (or the non-human correlate thereof). The method includes contacting a first physiological fluid sample from a mammal at risk of, suspected of having or having neurological disorders including but not limited to MLD and Alzheimer's disease and a substrate having one or more first moieties that specifically bind Tau, aggregates thereof, Abeta, ADDLs or globulimers, or complexes of Tau or aggregrates thereof, and Abeta, ADDLs or globulimers, which includes variants and fragments of Tau and Abeta, thereby forming a first complex. In one embodiment, the moieties that are employed in the method are antibodies specific for Tau, or for Abeta, including aggregates of Abeta such as small diffusible Abeta oligomers referred to as ADDLs (see U.S. Pat. No. 6,218,506 and Lambert et al., Proc. Natl. Acad. Sci., 95:6448 (1998); the disclosures of which are incorporated by reference herein) or globulomers (see U.S. published application 2009/0035307, WO 07/064,917 and Yu et al., Biochem., Structural Characterization of a Soluble Amyloid (3 peptide Oligomer, epub Feb. 13, 2009; the disclosures of which are incorporated by reference herein), or specific for complexes of Tau or aggregates thereof and Abeta or specific aggregates thereof such as ADDLs and globulomers, or combinations of those antibodies. In one embodiment, the physiological fluid is blood, e.g., blood serum. In one embodiment, if the sample contains the ligand for the one or more first moieties, the resulting complex maybe detected by contacting that complex with one or more second moieties that bind Abeta, ADDLs or globulomers (if the one or more first moieties bind Tau or aggregates thereof) or that bind Tau or aggregates thereof (if the one or more first moieties bind Abeta, ADDLs or globulimers), thereby forming a second complex. The amount of second complexes may be directly detected, e.g., the second moiety has a detectable label, such as a fluorescent label, or indirectly detected, e.g., the second moiety comprises a biotin label and that label is detected with a nanoparticle having streptavidin linked thereto. The amount of second complexes may be compared with the amount of uncomplexed Tau, aggregates of Tau, Abeta, ADDLs or globulomers in the physiological fluid sample. In one embodiment, the amount of second complexes is compared with second complexes formed by contacting a second physiological sample from the mammal from a different time point. In one embodiment, the mammal is a human. In one embodiment, the one or more moieties are specific for ADDLs. In one embodiment, the one or more moieties are specific for globulomers. In one embodiment, the one or more moieties are monoclonal antibodies which are employed to capture, immobilize or detect one of Tau, aggregates of Tau, Abeta, ADDLs or globulomers. In one embodiment, the one or more moieties are polyclonal antibodies employed to capture, immobilize or detect on of Tau, aggregates of Tau, Abeta, ADDLs or globulomers. In one embodiment, the one or more capture antibodies are specific for Tau or aggregates of Tau and the one or more detection antibodies are specific for Abeta, ADDLs or globulomers, e.g., the method detects complexes of Tau and Abeta, ADDLs or globulomers in physiological fluid. In another embodiment, the one or more capture antibodies are specific for Abeta, ADDLs or globulomers and the one or more detection antibodies are specific for Tau or aggregates of Tau e.g., the method detects complexes of Tau and Abeta, ADDLs or globulomers in physiological fluid. In one embodiment, the method detects complexes of Tau or aggregates thereof and ADDLs in physiological fluid. In another embodiment, the method detects complexes of Tau or aggregates thereof and globulomers in physiological fluid.

In one embodiment, the one or more capture antibodies are specific for Tau or aggregates of Tau and the one or more detection antibodies bind Abeta, ADDLs and globulomers, e.g., the method detects complexes of Tau and any of Abeta, ADDLs of globulomers in physiological fluid. In another embodiment, the one or more capture antibodies bind Abeta, ADDLs and globulomers and the one or more detection antibodies are specific for Tau or aggregates of Tau e.g., the method detects complexes of Tau and any of Abeta, ADDLs or globulomers in physiological fluid. For methods that may detect complexes as well as uncomplexed Tau or aggregates thereof, or Abeta, ADDLs and globulomers, a subtractive method may be employed to determine the amount of complexes of Tau and Abeta, ADDLs or globulomers.

In yet another embodiment, the one or more capture antibodies are specific for Tau or aggregates of Tau and the one or more detection antibodies are specific for Tau or aggregates of Tau, e.g., the method detects tau aggregates in physiological fluid.

In one embodiment, the method provides an assay that allows for diagnosis, prognosis, screening, staging, treatment monitoring, treatment planning or ruling out of neurological disorders including but not limited to MLD and Alzheimer's disease in a mammal, e.g., a human. In one embodiment, the first complexes are detected with one or more second moieties linked to a detectable molecule, such as a nanoparticle, an oligonucleotide or barcode. In one embodiment, to enhance the detection of the detectable molecule, the signal generated by the detectable molecule can be amplified. For instance, a silver coating (deposition) on a gold nanoparticle bound to a complex on a substrate can amplify the signal generated by the presence of the gold nanoparticle when exposed to light.

In one embodiment, a solid substrate comprises a plurality of different physically separated Tau, aggregates of Tau, Abeta, ADDLs or globulomers specific binding moieties, e.g., Tau, aggregates of Tau, Abeta, ADDLs or globulomers specific antibodies are each present at different preselected positions on the solid substrate. Contacting the solid substrate with a physiological sample can provide for a profile of the presence and/or amounts of Tau, aggregates of Tau, Abeta, ADDLs or globulomers, or complexes thereof. Those profiles may be useful for diagnosis, prognosis, staging, screening, selection of therapies, monitoring of therapy, or any combination thereof. Other factors which may be considered in the differential diagnosis, outcome or therapy selection include, but are not limited to, gender, ethnicity, age, as well as any other biomarker. In one embodiment, where the solid substrate comprises a first antibody specific for ADDLs, e.g., a monoclonal antibody, a polyclonal (second) antibody specific for Tau linked to a detectable molecule is employed to detect complexes of Tau and ADDLS in physiological fluid. In one embodiment, the second antibody with the detectable molecule is itself detected with a different detectable molecule, e.g., a biotin labeled polyclonal antibody is detected with streptavidin coated nanoparticles. In another embodiment, a solid substrate comprises an antigen as the first binding moiety, e.g., tau aggregates, and the second binding moiety comprises a polyclonal antibody specific for Tau and a detectable molecule. The polyclonal antibody with the detectable molecule itself may be detected with a different detectable molecule, e.g., a biotin labeled polyclonal antibody is detected with streptavidin coated nanoparticles.

In one embodiment, the sample is first contacted with the detection probe and then contacted with the capture probe. In another embodiment, the sample is first contacted with the capture probe and then contacted with the detection probe. In yet another embodiment, the sample, the detection probe, and the capture probe are contacted simultaneously.

In one embodiment, the nanoparticle is conjugated directly to the binding moiety. In another embodiment, the nanoparticle is conjugated indirectly to the binding moiety by a bridge or linker molecule. For example, the nanoparticle and binding moiety may each be conjugated to biotin and the nanoparticle and second binding moiety may be joined by an avidin or streptavidin bridge.

In one embodiment, the first binding moiety is bound to a substrate. For example, the substrate may be a nanoparticle, a thin film, or a magnetic bead. In one embodiment, the substrate has a planar surface. In illustrative embodiments, the substrate is made of glass, quartz, ceramic, or plastic. In some embodiments, the substrate is addressable.

In one embodiment, the complex is detected by photonic, electronic, acoustic, optoacoustic, gravitic, electro-chemical, electro-optic, mass-spectrometric, enzymatic, chemical, biochemical, magnetic, paramagnetic, or physical means. In one embodiment, the detecting step comprises contacting the substrate with silver stain. In one embodiment, the detecting comprises detecting light scattered by the nanoparticles.

In one embodiment, the nanoparticles are made of a noble metal, e.g., gold or silver. In one embodiment, the substrate is a nanoparticle, a thin film, or a magnetic bead. In one embodiment, the substrate has a planar surface and is made of glass, quartz, ceramic, or plastic. In some embodiments, the substrate is addressable.

Also included are methods for detecting Tau, Abeta, ADDLs or globulimers that are more sensitive, which employ a cutoff that may be used to differentiate one population or risk group from another.

Also provided is a computer-readable medium, with instructions thereon, which when executed by a processor of a computing device, cause the computing device to: receive one or more inputs indicative of detected amounts of complexes in physiological fluid samples taken from a test subject; evaluate the one or more inputs as a function of one or more algorithms stored on the computer-readable medium to diagnose, predict, screen for, stage, monitor treatment, provide for treatment planning, or rule out neurological disorders including but not limited to MLD and Alzheimer's disease for the test subject; and provide an output indicative of the diagnosis, prognosis, screening, staging, monitoring, treatments or rule out for the test subject.

Further provided is a system. The system includes a bus; a network interface coupled to the bus; a processor coupled to the bus; a memory coupled to the bus and holding an instruction set executable on the processor to receive, over the network interface from a client, one or more inputs indicative of detected amounts of Tau, aggregates of Tau, Abeta, ADDLs or globulomers, or complexes thereof, in a physiological fluid sample taken from a test subject; evaluate the inputs as a function of one or more algorithms held in the memory, the algorithms executable with regard to the inputs to diagnose, predict, screen for, stage, monitor treatment, provide for treatment planning for neurological disorders including but not limited to MLD and Alzheimer's disease of the test subject; and provide, to the client over the network interface, an output indicative of the diagnosis, prognosis, screen, stage, monitor, plan treatments, or rule out disease in the test subject.

The invention also provides a method that detects autoantibodies specific for Tau, aggregates of Tau, Abeta, ADDLs or globulomers, or complexes of Tau and Abeta, ADDLs or globulomers, in physiological fluid, e.g., blood or serum. In one embodiment, the detection of complexes of Tau and Abeta, ADDLs or globulomers, in physiological fluid is indicative of, for instance, neurological disorders including but not limited to MLD and Alzheimer's disease or a subject at risk of having neurological disorders including but not limited to MLD and Alzheimer's disease. In one embodiment, the invention provides a method for the diagnosis of neurological disorders including but not limited to MLD and Alzheimer's disease in a subject. The method includes providing a substrate having a capture probe bound thereto, wherein the capture probe comprises an antigen such as Tau, aggregates of Tau, Abeta, ADDLs or globulomers that is capable of specifically binding to complexes of Tau, aggregates of Tau, Abeta, ADDLs or globulomers bound to autoantibodies present in physiological fluid, such as blood; contacting the substrate having the capture probe bound thereto with a physiological fluid sample from the subject and a detection probe having a nanoparticle and a binding moiety that specifically binds to the autoantibody; and detecting the formation of the complex having the capture probe and detection probe. In one embodiment, the presence of the complex having the capture probe and detection probe is indicative of neurological disorders including but not limited to MLD and Alzheimer's disease in the subject.

In one embodiment, a method for detecting neurological disorders including but not limited to MLD and Alzheimer's disease-associated autoantibodies present in a physiological fluid sample from a subject is provided. The method includes contacting the sample with a capture probe, wherein the capture probe comprises a first binding moiety capable of specifically binding Tau, aggregates of Tau, Abeta, ADDLs or globulomers including variants thereof or peptides derived therefrom and a detection probe comprising a second binding moiety capable of specifically binding antibodies, e.g., of a particular isotype such as IgG, IgM, IgD, IgE or IgA; and detecting the presence of a complex formed between the capture probe, the disease-associated antigen bound to autoantibodies, and the detection probe.

In one embodiment, the first binding moiety is an antibody, antibody fragment, aptamer, or polypeptide. For example, the first binding moiety may be a polyclonal antibody specific for Tau, aggregates of Tau, Abeta, ADDLs or globulomers, variants thereof, or peptides derived from Tau, aggregates of Tau, Abeta, ADDLs or globulomers. Alternatively, the first binding moiety may be monoclonal antibody specific for Tau, aggregates of Tau, Abeta, ADDLs or globulomers, variants thereof, or peptides derived from Tau, aggregates of Tau, Abeta, ADDLs or globulomers. Binding a conserved region of the specific antigen followed by labeling autoantibodies attached to the antigen is a strategy for detection of variant forms of the antigen that may not be detectable with conventional sandwich assays, which would only recognize wild type forms of the antigen.

In one embodiment, the sample is first contacted with the detection probe and then contacted with the capture probe. In another embodiment, the sample is first contacted with the capture probe and then contacted with the detection probe. In yet another embodiment, the sample, the detection probe, and the capture probe are contacted simultaneously.

In one embodiment the binding moiety that specifically binds to the autoantibodies is an anti-human Ig antibody. For example, the anti-human antibody is selected from the group consisting of: anti-human IgG, anti-human IgM, anti-human IgA, anti-human IgE, anti-human IgD, and subtypes or mixtures thereof. In one embodiment, the detection probe further comprises a fluorophore, a phosphor, a quantum dot, an enzyme conjugate, or an avidin/biotin conjugate.

In one embodiment, the nanoparticle is conjugated directly to the binding moiety. In another embodiment, the nanoparticle is conjugated indirectly to the binding moiety by a bridge or linker molecule. For example, the nanoparticle and binding moiety may each be conjugated to biotin and the nanoparticle and second binding moiety may be joined by an avidin or streptavidin bridge.

In one embodiment, the first binding moiety is bound to a substrate. For example, the substrate may be a nanoparticle, a thin film, or a magnetic bead. In one embodiment, the substrate has a planar surface. In illustrative embodiments, the substrate is made of glass, quartz, ceramic, or plastic. In some embodiments, the substrate is addressable.

In one embodiment, the complex is detected by photonic, electronic, acoustic, optoacoustic, gravitic, electro-chemical, electro-optic, mass-spectrometric, enzymatic, chemical, biochemical, magnetic, paramagnetic, or physical means. In one embodiment, the detecting step comprises contacting the substrate with silver stain. In one embodiment, the detecting comprises detecting light scattered by the nanoparticles.

In one embodiment, the nanoparticles are made of a noble metal, e.g., gold or silver. In one embodiment, the substrate is a nanoparticle, a thin film, or a magnetic bead. In one embodiment, the substrate has a planar surface and is made of glass, quartz, ceramic, or plastic. In some embodiments, the substrate is addressable.

### BRIEF DESCRIPTION OF THE FIGURES

FIG. 1. A sandwich detection for amyloid-Tau complex.

FIG. 2. A detection format for Amyloid-Tau complex.

FIG. 3. A detection format for Autoantibody-Tau complex.

FIG. 4. A detection format for Tau aggregates.

### DETAILED DESCRIPTION

### Definitions

A “detectable moiety” is a label molecule attached to, or synthesized as part of, a polynucleotide. These detectable moieties include but are not limited to radioisotopes, colorimetric, fluorometric or chemiluminescent molecules, enzymes, haptens, redox-active electron transfer moieties such as transition metal complexes, metal labels such as silver or gold particles, or even unique oligonucleotide sequences.

A “biological sample” can be obtained from an organism, e.g., it can be a physiological fluid or tissue sample, such as one from a human patient, a laboratory mammal such as a mouse, rat, pig, monkey or other member of the primate family, by drawing a blood sample, sputum sample, spinal fluid sample, a urine sample, a rectal swab, a peri-rectal swab, a nasal swab, a throat swab, or a culture of such a sample. Thus, biological samples include, but are not limited to, whole blood or components thereof, blood or components thereof, blood or components thereof, semen, cell lysates, saliva, tears, urine, fecal material, sweat, buccal, skin, cerebrospinal fluid, and hair. Biological samples can be obtained from subjects for diagnosis or research or can be obtained from undiseased individuals, as controls or for basic research.

“Analyte” or “target analyte” is a substance to be detected in a test physiological sample using the present invention. The analyte can be any substance, e.g., a protein, or a set of related proteins, e.g., metabolites thereof.

“Capture moiety” is a specific binding member, capable of binding the analyte, which moiety may be in solution or directly or indirectly attached to a substrate. One example of a capture moiety includes an antibody bound to a support either through covalent attachment or by adsorption onto the support surface.

The term “ligand” refers to any organic compound for which a receptor or other binding molecule naturally exists or can be prepared. The term ligand also includes ligand analogs, which are modified ligands, usually an organic radical or analyte analog, usually of a molecular weight greater than 100, which can compete with the analogous ligand for a receptor, the modification providing means to join the ligand analog to another molecule. The ligand analog usually differs from the ligand by more than replacement of a hydrogen with a bond which links the ligand analog to another molecule, e.g., a label, but need not. The ligand analog can bind to the receptor in a manner similar to the ligand. The analog could be, for example, an antibody directed against the idiotype of an antibody to the ligand. For instance, a capture antibody may have a label that binds another molecule, e.g., the antibody is linked to biotin and strapetavidin is coated onto a substrate.

The term “receptor” or “antiligand” refers to any compound or composition capable of recognizing a particular spatial and polar organization of a molecule, e.g., epitopic or determinant site. Illustrative receptors include naturally occurring receptors, e.g., thyroxine binding globulin, antibodies, enzymes, Fab fragments, lectins, nucleic acids, avidin, protein A, barstar, complement component Clq, and the like. Avidin is intended to include egg white avidin and biotin binding proteins from other sources, such as streptavidin.

The term “antibody” refers to an immunoglobulin which specifically binds to and is thereby defined as complementary with a particular spatial and polar organization of another molecule, including recombinant antibodies such as chimeric antibodies and humanized antibodies. The antibody can be monoclonal or polyclonal and can be prepared by techniques that are well known in the art such as immunization of a host and collection of sera (polyclonal) or by preparing continuous hybrid cell lines and collecting the secreted protein (monoclonal), or by cloning and expressing nucleotide sequences or mutagenized versions thereof coding at least for the amino acid sequences required for specific binding of natural antibodies. Antibodies may include a complete immunoglobulin or fragment thereof, which immunoglobulins include the various classes and isotypes, such as IgA, IgD, IgE, IgG1, IgG2a, IgG2b and IgG3, IgM, etc. Fragments thereof may include Fab, Fv and F(ab′)₂, Fab′, and the like. In addition, aggregates, polymers, and conjugates of immunoglobulins or their fragments can be used where appropriate so long as binding affinity for a particular molecule is maintained.

The term “monoclonal antibody” as used herein refers to an antibody obtained from a population of substantially homogeneous antibodies, i.e., the individual antibodies comprising the population are identical except for possible naturally occurring mutations that may be present in minor amounts. For example, a monoclonal antibody can be an antibody that is derived from a single clone, including any eukaryotic, prokaryotic, or phage clone, and not the method by which it is produced. A monoclonal antibody composition displays a single binding specificity and affinity for a particular epitope. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. Monoclonal antibodies can be prepared using a wide variety of techniques known in the art including, e.g., but not limited to, hybridoma, recombinant, and phage display technologies. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The monoclonal antibodies may also be isolated from phage antibody libraries using the techniques described in Clackson et al., Nature 352:624-628 (1991) and Marks et al., J. Mol. Biol. 222:581-597 (1991), for example.

As used herein, the term “epitope related antibody” includes immunologically cross-reactive antibodies to homologs, metabolites, and variants of Tau, aggregates of Tau, Abeta, ADDLs or globulomers antigens associated with neurological disorders including but not limited to MLD and Alzheimer's disease. Epitope related antibodies may recognize functionally equivalent antigens seen in, e.g., (1) non human primates, rodents, canines, and other animal models; (2) derived tissue models, as well as (3) native or genetically engineered or assembled cellular assay models.

As used herein, the terms “immunologically cross-reactive” and “immunologicallyreactive” are used interchangeably to mean an antigen which is specifically reactive with an antibody which was generated using the same (“immunologically-reactive”) or different (“immunologically cross-reactive”) antigen.

As used herein, the term “immunologically-reactive conditions” means conditions which allow an antibody to bind to that epitope or a structurally similar epitope to a detectably greater degree than the antibody binds to substantially all other epitopes, generally at least two times above background binding, preferably at least five times above background. Immunologically-reactive conditions are dependent upon the format of the antibody binding reaction and typically are those utilized in immunoassay protocols. See, Harlow & Lane, Antibodies, A Laboratory Manual (Cold Spring Harbor Publications, New York (1988), for a description of immunoassay formats and conditions.

As used herein, the term “array” refers to a population of different molecules (e.g., capture probes) that are attached to one or more substrates such that the different probe molecules can be differentiated from each other according to relative location. An array can include different probe molecules that are each located at a different addressable location on a substrate. Alternatively, an array can include separate substrates each bearing a different probe molecule. Probes attached to separate substrates can be identified according to the locations of the substrates on a surface to which the substrates are associated or according to the locations of the substrates in a liquid. As used herein, the term “addressable array” or “addressable substrate” refers to an array wherein the individual elements have precisely defined coordinates, so that a given element at a particular position in the array can be identified.

The term “antigen” refers to is a substance that prompts the generation of antibodies and can cause an immune response. Examples of antigens include, but are not limited to, Tau, aggregates of Tau, Abeta, ADDLs or globulomers, variants or fragments thereof, that are immunologically reactive or cross-reactive with antibodies specific therefor or autoantibodies present in the blood or components thereof.

As used herein, the term “disease-associated antigen,” refers to a substance associated with a disease or medical condition in a subject, e.g., neurological disorders including but not limited to MLD and Alzheimer's disease, resulting in the production of autoantibodies. Disease-associated antigens include the wildtype protein, complexes, and aggregates as well as modified forms (mutants, haplotypes, or other variant forms), complexes, and aggregates of wild-type proteins.

As used herein, the term “antibody” means a polypeptide comprising a framework region from an immunoglobulin gene or fragments thereof that specifically binds and recognizes an antigen. Use of the term antibody is meant to include whole antibodies, including singlechain whole antibodies, antibody fragments such as Fab fragments, and other antigen-binding fragments thereof. The term “antibody” includes bispecific antibodies and multispecific antibodies so long as they exhibit the desired biological activity or function.

As used herein, the term “polyclonal antibody” means a preparation of antibodies derived from at least two (2) different antibody-producing cell lines. The use of this term includes preparations of at least two (2) antibodies that contain antibodies that specifically bind to different epitopes or regions of an antigen.

An “autoantibody” (abbreviated “autoantibody”) is an antibody produced by the immune system of a subject that is directed against one or more of the subject's own proteins.

As used herein, the term “binding agent” or “binding moiety” is a compound, a macromolecule, including polypeptide, DNA, RNA and carbohydrate that selectively binds a target molecule. For example, a binding agent can be a polypeptide that selectively binds with high affinity or avidity to a target analyte without substantial cross-reactivity with other polypeptides that are unrelated to the target analyte. The affinity of a binding agent that selectively binds a target analyte will generally be greater than about 10-5 M, such as greater than about 10-6 M, including greater than about 10-8 M and greater than about 10-9 M. Specific examples of such selective binding agents include a polyclonal or monoclonal antibody specific for a disease-associated antigen or human immunoglobulin. The binding agent can be labeled with a detectable moiety, if desired, or rendered detectable by specific binding to a detectable secondary binding agent.

As used herein, the term “capture probe” refers to a molecule capable of binding to a target analyte, e.g., a disease-associated autoantibody. One example of a capture probe includes antigens that recognize autoantibodies present in a biological sample from patients having or suspected of having a disease, e.g., neurological disorders including but not limited to MLD and Alzheimer's disease. Other examples of capture probes include aptamers, protein ligands, etc., which are described for instance, in PCT/US01/10071 (Nanosphere, Inc.).

As used herein, the term “complex” means an aggregate of two or more molecules that result from specific binding between the molecules, such as an antibody and an antigen, a receptor and a ligand, and the like.

A “detection probe” is a labeled molecule including one or more binding agents, wherein the one or more binding agents specifically bind to a specific target analyte. The label itself may serve as a carrier, or the probe may be modified to include a carrier. Carriers that are suitable for the methods include, but are not limited to, nanoparticles, quantum dots, dendrimers, semi-conductors, beads, up- or down-converting phosphors, large proteins, lipids, carbohydrates, or any suitable inorganic or organic molecule of sufficient size, or a combination thereof.

The term “homology” refers to sequence similarity between two peptides or between two nucleic acid molecules. Homology may be determined by comparing a position in each sequence, which may be aligned for purposes of comparison. When a position in the compared sequence is occupied by the same base or amino acid, then the molecules are homologous at that position. A degree of homology between sequences is a function of the number of matching or homologous positions shared by the sequences.

“Identity” means the degree of sequence relatedness between polypeptide or polynucleotide sequences, as the case may be, as determined by the match between strings of such sequences. “Identity” and “homology” can be readily calculated by known methods. Suitable computer program methods to determine identity and homology between two sequences include, but are not limited to, the GCG program package (Devereux, J., et al., Nucleic Acids Research 12(1): 387 (1984)), BLASTP, BLASTN, and FASTA (Atschul, S. F. et al., J. Molec. Biol. 215: 403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990).

As used herein, the terms “label” or “detectable label” refers to a marker that may be detected by photonic, electronic, opto-electronic, magnetic, gravitic, acoustic, enzymatic, magnetic, paramagnetic, or other physical or chemical means. The term “labeled” refers to incorporation of such a detectable marker, e.g., by incorporation of a radiolabeled molecule or attachment to a nanoparticle.

As used herein, the term “level” is intended to mean the amount, accumulation or rate of synthesis of a molecule. The term can be used to refer to an absolute amount of a molecule in a sample or to a relative amount of the molecule, including amounts determined under steady state or non-steady-state conditions. The level of a molecule can be determined relative to a control molecule in a sample. The level of a molecule also can be referred to as an expression level.

The term “ortholog” refers to genes or proteins which are homologs via speciation, e.g., closely related and assumed to have common descent based on structural and functional considerations. Orthologous proteins function as recognizably the same activity in different species. The term “paralog” denotes a polypeptide or protein obtained from a given species that has homology to a distinct polypeptide or protein from that same species.

As used herein, the term “reference level” is intended to mean a control level of a biomarker, e.g., disease-associated autoantibody, used to evaluate a test level of the biomarker in a sample from an individual. A reference level can be a normal reference level or a disease-state reference level. A normal reference level is an amount of expression of a biomarker in a non-diseased subject or subjects. A disease-state reference level is an amount of expression of a biomarker in a subject with a positive diagnosis for the disease or condition. A reference level also can be a stage-specific reference level. A stage-specific reference level refers to a level of a biomarker characteristic of a given stage of progression of a disease or condition.

The term “specific binding” refers to that binding which occurs between such paired species as enzyme/substrate, receptor/agonist, antibody/antigen, and lectin/carbohydrate which may be mediated by covalent or non-covalent interactions or a combination of covalent and noncovalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs. Thus, for example, an antibody typically binds to a single epitope and to no other epitope within the family of proteins. In some embodiments, specific binding between an antigen and an antibody will have a binding affinity of at least 10-6 M. In other embodiments, the antigen and antibody will bind with affinities of at least 10-7 M, 10-8 M to 10-9 M, 10-10 M, 10-11 M, or 10-12M.

As used herein the phrase “splice variant” refers to mRNA molecules produced from primary RNA transcripts that have undergone alternative RNA splicing. Alternative RNA splicing occurs when a primary RNA transcript undergoes splicing, generally for the removal of introns, which results in the production of more than one mRNA molecule each of which may encode different amino acid sequences. The term “splice variant” also refers to the proteins encoded by the above mRNA molecules.

As used herein, the term “subject” means the subject is a mammal, such as a human, but can also be an animal, e.g., domestic animals (e.g., dogs, cats and the like), farm animals (e.g., cows, sheep, pigs, horses and the like) and laboratory animals (e.g., monkey, rats, mice, rabbits, guinea pigs and the like).

As used herein, the term “substitution” is one of mutations that is generally used in the art. Substitution variants have at least one amino acid residue in a polypeptide molecule replaced by a different residue. “Conservative substitutions” typically provide similar biological activity as the unmodified polypeptide sequence from which the conservatively modified variant was derived. Conservative substitutions typically include the substitution of one amino acid for another with similar characteristics. Conservative substitution tables providing functionally similar amino acids are well known in the art. For example, the following six groups each contain amino acids that are conservative substitutions for one another: Aliphatic: Glycine (G), Alanine (A), Valine (V), Leucine (L), Isoleucine (I); Aromatic: Phenylalanine (F), Tyrosine (Y), Tryptophan (W); Sulfur-containing: Methionine (M), Cysteine (C); Basic (Cationic): Arginine (R), Lysine (K), Histidine (H); Acidic (Anionic): Aspartic acid (D), Glutamic acid (E); Amide: Asparagine (N), Glutamine (Q).

As used herein, the term “substrate” refers to any surface capable of having capture probes bound thereto. Such surfaces include, but are not limited to, glass, metal, plastic, or materials coated with a functional group designed for binding of capture probes or analytes. Substrates also may be referred to as slides.

As used herein, the terms “treating,” “treatment,” or “alleviation” refers to both therapeutic treatment and prophylactic or preventative measures, wherein the object is to prevent or slow down (lessen) the targeted pathologic condition or disorder. A subject is successfully “treated” for a disorder characterized by increased autoantibody levels if the subject shows observable and/or measurable reduction in or absence of one or more signs and symptoms of a particular disease or condition.

As used herein, the term “variant polypeptide” refers to a polypeptide that differs from a naturally occurring polypeptide in amino acid sequence or in ways that do not involve amino acid sequence modifications, or both. Non-sequence modifications include, but are not limited to, changes in citrullination, acetylation, methylation, phosphorylation, carboxylation, or glycosylation.

Variants may also include sequences that differ from the wild-type sequence by one or more amino acid substitutions, deletions, or insertions. The term “allelic variant” denotes any of two or more alternative forms of a gene occupying the same chromosomal locus. Allelic variation arises naturally through mutation, and may result in phenotypic polymorphism within populations. Gene mutations can be silent (no change in the encoded polypeptide) or may encode polypeptides having altered amino acid sequence. The term allelic variant is also used herein to denote a protein encoded by an allelic variant of a gene.

In the description that follows, a number of terms are utilized extensively. Definitions are herein provided to facilitate understanding of the invention. The terms described below are more fully defined by reference to the specification as a whole. In practicing the invention, many conventional techniques in molecular biology, protein biochemistry, cell biology, immunology, microbiology and recombinant DNA are used. These techniques are well-known and are explained in, e.g., Current Protocols in Molecular Biology, Vols. I-III, Ausubel, Ed. (1997); Sambrook et al., Molecular Cloning: A Laboratory Manual, Second Ed. (Cold Spring Harbor Laboratory Press, Cold Spring Harbor, N.Y. (1989)); DNA Cloning: A Practical Approach, Vols. I and II, Glover, Ed. (1985); Oligonucleotide Synthesis, Gait, Ed. (1984); Nucleic Acid Hybridization, Hames & Higgins, Eds. (1985); Transcription and Translation, Hames & Higgins, Eds. (1984); Animal Cell Culture, Freshney, Ed. (1986); Immobilized Cells and Enzymes (IRL Press (1986)); Perbal, A Practical Guide to Molecular Cloning; the series, Meth. Enzymol., (Academic Press, Inc. (1984)); Gene Transfer Vectors for Mammalian Cells, Miller & Calos, Eds. (Cold Spring Harbor Laboratory, NY (1987); and Meth. Enzymol., Vols. 154 and 155, Wu & Grossman, and Wu, Eds., respectively. Units, prefixes, and symbols may be denoted in their accepted SI form.

Unless defined otherwise, all technical and scientific terms used herein generally have the same meaning as commonly understood by one of ordinary skill in the art to which this invention belongs. As used in this specification and the appended claims, the singular forms “a,” “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well known and commonly employed in the art.

### Methods of the Invention

The invention provides sensitive methods to detect the presence or amount of Tau, or aggregates thereof, Abeta, ADDLs or globulomers including variants thereof or peptides derived therefrom in a sample. In one embodiment, the levels of Tau, or aggregates thereof, Abeta, ADDLs or globulomers including variants thereof or peptides derived therefrom in a patient physiological sample, e.g., a physiological fluid sample, such as blood plasma, blood serum or saliva, or a tissue biopsy, e.g., are tested.

In one embodiment, one or more different types of capture moieties that bind to Tau, Abeta, ADDLs or globulomers including variants thereof or peptides derived therefrom may be immobilized onto the surface of a substrate, e.g., before contact with the sample. The capture moiety may be bound to the substrate by any conventional means including one or more linkages between the capture probe and the surface or by adsorption. In one embodiment, one or more different types of capture moieties that bind to Tau, Abeta, ADDLs or globulomers including variants thereof or peptides derived therefrom are contacted with the sample and in one embodiment, the resulting complex is immobilized onto the surface of a substrate. In another embodiment, the complex is not immobilized onto a substrate. The capture moiety and the ligand therefor in the sample may be specific binding pairs such as antibody-antigen or receptor-ligand, or may be subunits of a macromolecule such as an aggregate of tau molecules, which aggregate may be formed of nonidentical tau molecules (a heterogeneous population of tau molecules). The presence of any target analyte-capture moiety complex is then detected, e.g., using probes having a detectable molecule. In one embodiment, selection of various Tau, Abeta, ADDLs or globulomers specific antibodies, e.g., antibodies specific for different forms of Tau or Abeta, or for more than one form, may be employed as capture or detection moieties.

In one embodiment, where the detectable molecule is a nanoparticle, the presence of the nanoparticle may be detected by flow-based methods or detection may be enhanced by silver staining Silver staining can be employed with any type of nanoparticle that catalyzes the reduction of silver. In one embodiment, the nanoparticles are made of noble metals (e.g., gold and silver). See Bassell et al., J. Cell Biol., 126:863 (1994); Braun-Howland et al., Biotechniques, 13:928 (1992). Silver staining has been found to provide a large increase in sensitivity for assays employing a single type of nanoparticle. For greater enhancement of the detectable change, one or more layers of nanoparticles may be used, each layer treated with silver stain as described in PCT/US01/21846.

In one embodiment, detection may employ a silver-amplified antibody probe array, a biobarcode assay, or a flow-based detection of nanoparticles (see, e.g., Nam et al., Science, 301:1884 (2003); Bao et al., Anal. Chem., 78:2055 (2006); U.S. Pat. Nos. 7,110,585; 6,506,564; 6,602,669; 6,645,721; 6,673,548; 6,677,122; 6,720,147; 6,730,269; 6,750,016; 6,767,702; 6,759,199; 6,812,334; 6,818,753; 6,903,207; 6,962,786; and 6,986,989, all of which are incorporated herein by reference). In these approaches, a solid substrate such as a microarray slide, magnetic bead, microwell plate or test tube is functionalized with different specific capture moieties (e.g., monoclonal antibodies) capable of specifically capturing the target or form of interest, e.g., Tau-ADDL complexes. A sample is allowed to contact the substrate for variable times which enables different levels of target detection. Once captured, detection probes functionalized with complementary moieties capable of specific and defined attachment to the captured target or complexes that include the target, are introduced into the assay (note variations of this principle that are well established also can be used, including biotin-streptavidin interactions). Once this attachment is complete the signal for each captured target or form of interest may be amplified by silver deposition on captured gold probe (array-based assay), unique reporter biobarcode oligos are released and detected on an array (biobarcode assay) or variable encoded probes are released and detected by laser-based flow. The assay results are read by a detection system (e.g., VerigenelD or a Tecan scanner) and an algorithm determines the quantity of each individual moiety and calculates the relative and total results.

Neurological disorders including but not limited to MLD and Alzheimer's disease-associated marker proteins may be found both in the tissues and in the bodily fluids of an individual who suffers from that disease. The levels may be very low at the early stages of the disease process and increase during progression of the disease or first increase then decrease as the disease progresses. Autoantibodies produced by patients suffering from neurological disorders including but not limited to MLD and Alzheimer's disease may specifically recognize neurological disorders including but not limited to MLD and Alzheimer's disease associated marker proteins, such as Tau, Abeta, ADDLs or globulomers including variants thereof or peptides derived therefrom, or complexes thereof. The detection of Tau, or aggregates thereof, Abeta, ADDLs or globulomers including variants thereof or peptides derived therefrom, including complexes thereof, and autoantibodies to Tau, or complexes thereof, Abeta, ADDLs or globulomers including variants thereof or peptides derived therefrom, or complexes thereof, in patients with disease may therefore be used to better diagnose, predict, screen for, stage, monitor treatment, provide for treatment planning, or rule out disease in an individual.

### Diagnostic Methods

The development of immunologic responsiveness to self is called autoimmunity and reflects the impairment of self-tolerance. Immunologic, environmental, and genetic factors are closely interrelated in the pathogenesis of autoimmunity. The frequency of autoimmune antibodies (autoantibodies) in the general population increases with age, suggesting a breakdown of self tolerance with aging. Autoantibodies also may develop as an aftermath of disease tissue damage.

The development of autoimmunity usually involves the breakdown or circumvention of self-tolerance. The potential for the development of autoantibodies probably exists in most individuals. For example, normal human B cells are capable of reacting with several self-antigens, but are suppressed from producing autoantibodies by one or more tolerance mechanisms. Precommitted B cells in tolerant individuals can be stimulated in several ways. For example, tolerance involving only T cells, induced by persistent low levels of circulating self-antigens, may breakdown in the presence of substances such as endotoxin. Such substances stimulate the B cells directly to produce autoantibodies. Another tolerance mechanism involves suppressor T cells. A decrease in suppressor T cell activity therefore may also lead to production of autoantibodies.

In various embodiments, the methods described herein may be used to detect autoantibodies raised against antigens associated with neurological disorders including but not limited to MLD and Alzheimer's disease. A disease-associated antigen may be a variant form of a polypeptide, i.e., a polypeptide formed as the result of mutation or alternative post-transitional modification. Such variants are also referred to herein as “neopeptides.” A number of antigens associated with neurological disorders including but not limited to MLD and Alzheimer's disease have been described in the literature. Some antigens associated with neurological disorders including but not limited to MLD and Alzheimer's disease are well characterized biochemically and by their antigenic character.

In one aspect, the disclosure provides methods of detecting autoantibodies associated with Alzheimer's diseaes in biological samples. In one embodiment, the method comprises contacting a sample with a capture probe comprising an antigen recognized by the target analytes (e.g., autoantibodies) and nanoparticles having anti-human Ig antibodies attached thereto. For example, the capture probe can bind to the antigen that is bound to an autoantibody and the nanoparticle probe comprising a detection antibody can bind to the antibody which is an autoantibody, thereby forming a sandwich complex. The presence, absence, and/or amount of the complex may be detected, wherein the presence or absence of the complex is indicative of the presence, absence, or amount of the autoantibodies. As described above, certain autoantibodies are biomarkers for neurological disorders including but not limited to MLD and Alzheimer's disease.

In a suitable embodiment, the method comprises using a sandwich assay to detect the autoantibodies. Sandwich assays generally involve the use of binding molecules (e.g., antibodies), each capable of binding to a different immunogenic portion, or epitope, of the protein or complex of biomolecules to be detected and/or quantitated. In a sandwich assay, the analyte (which may be a complex of heterogenous molecules) is typically bound by a first binding molecule which is immobilized on a solid support, and thereafter a second binding molecule binds to the analyte, thus forming an insoluble three part complex. See, e.g., U.S. Pat. No. 4,376,110. In some embodiments of these methods, the first binding molecule is an antigen, e.g., one that forms aggregates, the analyte is the antigen or aggregate bound to the autoantibody, and the second binding molecule is an anti-human Ig antibody which specifically binds to the autoantibody.

In one embodiment, the sample is first contacted with the detection probe so that an autoantibody present in the sample binds to the binding agent on the detector probe, and the autoantibody bound to the detection probe is then contacted with the substrate having capture probes bound thereto. In another embodiment, the sample is first contacted with the substrate so that autoantibodies complexed with an antigen present in the sample bind to a capture probe, and the autoantibodies complexed with the antigen bound to the capture probe are then contacted with the detection probe so that the antigen binds to the binding agent on the detection probe. In another embodiment, the sample, the detection probe and the capture probe on the substrate are contacted simultaneously.

An exemplary method for detecting the presence, absence, and/or amount of autoantibodies in a biological sample involves obtaining a biological sample (e.g., blood or components thereof, blood or components thereof or blood or components thereof) from a test subject and contacting the biological sample with an antigen recognized by autoantibodies such that the presence of the autoantibodies is detected in the biological sample. In one embodiment, the sample is first contacted with the substrate so that autoantibodies complexed with an antigen present in the sample bind to a capture probe, and the autoantibodies complexed with the antigen bound to the capture probe are then contacted with the detection probe so that the antigen binds to the binding agent on the detection probe. In another embodiment, the sample is first contacted with the detection probe so that the autoantibody present in the sample binds to the binding agent on the detector probe, and the autoantibody complexed with the antigen bound to the detection probe is then contacted with the substrate having capture probes specific for the antigen bound thereto. The amount of binding is compared with a suitable reference sample or control, which can be the amount of binding in the absence of the autoantibodies, the amount of the binding in the presence of a non-specific immunoglobulin composition, or both.

In some embodiments, the antigens recognized by the autoantibodies, when used in a sandwich assay employing gold-nanoparticle detection with silver enhancement, significantly improves the LOD for autoantibodies by lowering the detectable concentration of the complex formed between the antigen and the captured antibody. Additionally, in some embodiments, the assay employs a mixed set of biotinylated secondary antibody isotypes which allow more favorable detection of the response of human anti-antibodies—particularly a mixture of IgG, IgM, IgE, IgD, and IgA and subtypes thereof may be used as detection antibodies.

In other embodiments, the invention provides methods including contacting a sample with a capture probe comprising a first moiety that binds a target analyte such as Tau, or aggregates thereof, Abeta, ADDLs or globulomers including variants thereof or peptides derived therefrom, or complexes thereof, and a detection probe comprising a second moiety that binds Tau, or aggregates thereof, Abeta, ADDLs or globulomers including variants thereof or peptides derived therefrom, or complexes thereof, wherein in one embodiment the detection probe binds a different molecule than the first moiety, such as a different molecule found in the complexes. The detection probe may also include a detectable molecule, e.g., a nanoparticle or other molecule that binds a ligand. In one embodiment, the detection probe comprises a ligand and the detection probe is detected using a nanoparticle comprising a binding partner for the ligand. For example, the capture probe can bind to ADDLs that are bound to Tau molecules in the sample and the detection probe comprises anti-Tau antibodies bound to biotin, which are detected with a nanoparticle comprising streptavidin, thereby forming a sandwich complex. The presence, absence, and/or amount of the complex may be detected, wherein the presence or absence of the complex is indicative of the presence, absence, or amount of complexes of Tau or aggregates thereof and ADDLs. In a suitable embodiment, the method comprises using a sandwich assay to detect the complexes.

In one embodiment, the sample is first contacted with the detection probe and the resulting complex is then contacted with the capture probe. In another embodiment, the sample is first contacted with the substrate having the capture probe, and then contacted with the detection probe. In another embodiment, the sample, the detection probe and the capture probe on the substrate are contacted simultaneously.

Thus, the invention also provides a diagnostic method for neurological disorders including but not limited to MLD and Alzheimer's disease, which involves: assaying the levels of autoantibodies specific for Tau, or aggregates thereof, Abeta, ADDLs or globulomers including variants thereof or peptides derived therefrom or complexes thereof, or the levels of complexes of Tau, or aggregates thereof, and Abeta, ADDLs or globulomers, including variants thereof or peptides derived therefrom; and (b) comparing the amount of the autoantibodies or complexes of Tau, or aggregates thereof, and Abeta, ADDLs or globulomers, including variants thereof or peptides derived therefrom, with a reference standard, whereby an increase or decrease in the assayed autoantibodies or complexes of Tau, or aggregates thereof, and Abeta, ADDLs or globulomers, including variants thereof or peptides derived therefrom, compared to the standard level is indicative of a medical condition, i.e., neurological disorders including but not limited to MLD and Alzheimer's disease.

Reference Levels.

The reference level used for comparison with the measured level for an autoantibody or complexes may vary, depending on the aspect of the invention being practiced, as will be understood from the foregoing discussion. For disease diagnostic methods, the “reference level” is typically a predetermined reference level, such as an average of levels obtained from a population that is not afflicted with neurological disorders including but not limited to MLD and Alzheimer's disease, but in some instances, the reference level can be a mean or median level from a group of individuals including diseased patients. In some instances, the predetermined reference level is derived from (e.g., is the mean or median of) levels obtained from an agematched population. Alternatively, the reference level may be a historical reference level for the particular patient (e.g., an autoantibody level that was obtained from a sample derived from the same individual, but at an earlier point in time).

For disease staging or stratification methods (i.e., methods of classifying diseased patients into mild, moderate and severe stages of disease), the reference level is normally a predetermined reference level that is the mean or median of levels from a population which has been diagnosed with disease. In some instances, the predetermined reference level is derived from (e.g., is the mean or median of) levels obtained from an age-matched population.

Age-matched populations (from which reference values may be obtained) are ideally the same age as the individual being tested, but approximately age-matched populations are also acceptable. Approximately age-matched populations may be within 1, 2, 3, 4, or 5 years of the age of the individual tested, or may be groups of different ages which encompass the age of the individual being tested. Approximately age-matched populations may be in 2, 3, 4, 5, 6, 7, 8, 9, or 10 year increments (e.g., a “5 year increment” group which serves as the source for reference values for a 62 year old individual might include 58-62 year old individuals, 59-63 year old individuals, 60-64 year old individuals, 61-65 year old individuals, or 62-66 year old individuals).

Comparing Levels of Disease-Associated Autoantibodies or Complexes.

The process of comparing a measured value and a reference value can be carried out in any convenient manner appropriate to the type of measured value and reference value for the disease-associated antigen, complexes or autoantibody at issue. Measuring can be performed using quantitative or qualitative measurement techniques, and the mode of comparing a measured value and a reference value can vary depending on the measurement technology employed. For example, when a qualitative assay is used to measure disease-associated antigen, complexes or autoantibody levels, the levels may be compared by comparing data from densitometric or spectrometric measurements (e.g., comparing numerical data or graphical data, such as bar charts, derived from the measuring device). However, it is expected that the measured values used in the methods of the invention will most commonly be quantitative values (e.g., quantitative measurements of signal intensity).

A measured value is generally considered to be substantially equal to or greater than a reference value if it is at least 95% of the value of the reference value (e.g., a measured value of 1.71 would be considered substantially equal to a reference value of 1.80). A measured value is considered less than a reference value if the measured value is less than 95% of the reference value (e.g., a measured value of 1.7 would be considered less than a reference value of 1.80). A measured value is considered more than a reference value if the measured value is at least more than 5% greater than the reference value (e.g., a measured value of 1.89 would be considered more than a reference value of 1.80).

The process of comparing may be manual (such as visual inspection by the practitioner of the method) or it may be automated. For example, an assay device may include circuitry and software enabling it to compare a measured value with a reference value for a disease-associated antigen, complexes or autoantibody. Alternatively, a separate device (e.g., a digital computer) may be used to compare the measured value(s) and the reference value(s). Automated devices for comparison may include stored reference values for the disease-associated antigen, complexes or autoantibody being measured, or they may compare the measured value(s) with reference values that are derived from contemporaneously measured reference samples.

In some embodiments, the methods of the invention utilize “simple” or “binary” comparison between the measured level(s) and the reference level(s) (e.g., the comparison between a measured level and a reference level determines whether the measured level is higher or lower than the reference level). For example, for autoantibody levels, a comparison showing that the measured value for the autoantibody is higher than the reference value may indicate or suggest a diagnosis of neurological disorders including but not limited to MLD and Alzheimer's disease. It is useful to determine appropriate partitioning of data by performing a ROC analysis. A ROC curve is a plot of the true positive rate against the false positive rate for the different possible thresholds of a diagnostic test, wherein the threshold is related to the responses of the signals from said assays. This provides a method of measuring the clinical sensitivity and specificity of a specific subset of data or the data as a whole group. In one embodiment, a variable which may be useful (a positive variable, e.g., one with a statistically relevant predictive value) as a predictor for the group as a whole may become negative (statistically irrelevant as a predictor) after partitioning. Alternatively, a variable that is of negative value for a larger group may become a positive variable after partitioning, e.g., a positive variable to one of the groups resulting from partitioning. In one embodiment, a partition or other algorithm which employs data with regard to the amount of tau in blood, complexes of tau and Abeta in blood or autoantibodies to tau or complexes of tau and Abeta in blood, or combinations thereof, as well as other biomarkers or indicia of disease, is employed.

In certain aspects, the comparison is performed to determine the magnitude of the difference between the measured and reference values (e.g., comparing the “fold” or percentage difference between the measured value and the reference value). A fold difference that is about equal to or greater than the minimum fold difference disclosed herein suggests or indicates a diagnosis of a disease or medical condition, as appropriate to the particular method being practiced. A fold difference can be determined by measuring the absolute concentration of the disease-associated antigen, complex or autoantibody and comparing that to the absolute value of a reference, or a fold difference can be measured by the relative difference between a reference value and a 20 sample value, where neither value is a measure of absolute concentration, and/or where both values are measured simultaneously.

As will be apparent to those of skill in the art, when replicate measurements are taken for a specific molecule tested, the measured value that is compared with the reference value is a value that takes into account the replicate measurements. The replicate measurements may be taken into account by using either the mean or median of the measured values as the “measured value.

Multiple Marker Analysis for Subject Rule-in and Rule-Out

While assays using a single capture probe are informative, e.g., in the diagnosis of disease, combining the information from two or more capture probes into one algorithm can make a substantial improvement in the prediction. By optimizing the combined information, it is possible to increase the specificity and sensitivity of the assay.

More specifically, methods of predicting whether a patient has a specific disease or stage of disease can be improved by determining the quantity of two or more markers, including the quantity of complexes, autoantibodies or antigens disclosed herein, in a sample obtained from a patient against multiple other antigens. The data collected from the two or more measurements is subjected to statistical analyses wherein the quantity of autoantibody(s) or antigen(s) present in a sample is compared or normalized to a reference set of non-diseased samples enabling the determination of whether a specific disease is present, or alternatively, determining what stage of disease (i.e., disease progression or regression).

In a particular embodiment, the quantities obtained from the measurements are analyzed in multidimensional space (the dimensions of which comprise the responses of the signals from each of the separate assays), and the presence or absence of disease is determined by partitioning the signals on the basis of signal intensity from two or more of the measurements. It is useful to determine appropriate partitioning of data by performing a ROC analysis. A ROC curve is a plot of the true positive rate against the false positive rate for the different possible thresholds of a diagnostic test, wherein the threshold is related to the responses of the signals from said assays. This provides a method of measuring the clinical sensitivity and specificity of a specific subset of data or the data as a whole group. The two or more measurements may consist of measuring variants of antigens or autoantibodies present in a sample with different capture agents (e.g., different antigen and/or different x-human Ig antibodies, e.g., anti-IgM versus anti-IgG antibodies. The difference between the presence or amount of certain complexes, antigens or anti-IgM and anti-IgG antibodies may provide information regarding the stage of disease.

### Prognostic or Predictive Assays

The disclosure also provides for prognostic (or predictive) assays for determining whether an individual is at risk of developing a condition, disorder or disease associated with the presence or absence of certain complexes, antigens and/or autoantibodies. Such assays can be used for prognostic or predictive purpose, for example to thereby prophylactically treat an individual prior to the onset of a disorder characterized by or associated with autoantibodies or antigens, e.g., neurological disorders including but not limited to MLD and Alzheimer's disease. The methods described herein can also be used to determine the levels of such complexes, antigens and/or autoantibodies in subjects to aid in predicting the response of such subjects to medication. Another aspect of the invention provides methods for determining complexes, antigens and/or autoantibody profiles in an individual to thereby select appropriate therapeutic or prophylactic compounds for that individual.

Accordingly, the prognostic assays described herein can be used to determine whether a subject can be administered a compound (e.g., an agonist, antagonist, peptidomimetic, polypeptide, peptide, nucleic acid, small molecule, or other drug candidate) to treat a disease or condition associated with the presence of certain complexes, antigens and/or autoantibodies. Thus, the invention provides methods for determining whether a subject can be effectively treated with a compound for a disorder or condition associated with an aberrant complex, antigen and/or autoantibody levels or in which a test sample is obtained and the complexes, antigens and/or autoantibodies are detected using the assays described herein (e.g., wherein the presence, absence, and/or amount of the complexes, antigens and/or autoantibodies is diagnostic for a subject that can be administered the compound to treat a disorder associated with an aberrant complexes, antigens and/or autoantibody level).

For example, the level of the autoantibodies in a sample obtained from a subject is determined and compared with the level found in a obtained from a different subject (or population of subjects) who is free of the condition, in an earlier or later stage of the condition, has a more or less severe form of the condition or responds differently to treatments of the condition. An overabundance (or under abundance) of the autoantibodies in the sample obtained from the subject suspected of having the condition affecting autoantibody levels compared with the sample obtained from the different subject or population is indicative of the condition in the subject being tested.

The methods described herein can be performed, e.g., by utilizing pre-packaged diagnostic kits comprising at least one probe reagent, which can be conveniently used, e.g., in clinical settings diagnosis or prognosis subjects exhibiting symptoms of the condition.

Correlating a Subject to a Standard Reference Population.

To deduce a correlation between clinical response to a treatment and a particular level of complexes, antigens and/or autoantibodies, it is necessary to obtain data on the clinical responses exhibited by a population of individuals who received the treatment, i.e., a clinical population. This clinical data maybe obtained by retrospective analysis of the results of a clinical trial(s). Alternatively, the clinical data may be obtained by designing and carrying out one or more new clinical trials. The analysis of clinical population data is useful to define a standard reference population(s) which, in turn, are useful to classify subjects for clinical trial enrollment or for selection of therapeutic treatment. In one embodiment, the subjects included in the clinical population have been graded for the existence of the medical condition of interest. Grading of potential subjects can include, e.g., a standard physical exam or one or more lab tests. Alternatively, grading of subjects can include use of a biomarker expression pattern. For example, autoantibody level is a useful as grading criteria where there is a strong correlation between expression pattern and susceptibility or severity to a disease or condition. In one embodiment, a subject is classified or assigned to a particular group or class based on similarity between the measured levels of autoantibody in the subject and the level of the autoantibody observed in a standard reference population.

In one embodiment, a treatment of interest is administered to each subject in a trial population, and each subject's response to the treatment is measured using one or more predetermined criteria. It is contemplated that in many cases, the trial population will exhibit a range of responses, and that the investigator will choose the number of responder groups (e.g., low, medium, high) made up by the various responses. In addition, the expression level of a biomarker (e.g., complexes, autoantibodies or antigens) is quantified, which may be done before and/or after administering the treatment. These results are then analyzed to determine if any observed variation in clinical response between groups is statistically significant. Statistical analysis methods, which may be used, are described in L. D. Fisher & G. vanBelle, Biostatistics: A Methodology for the Health Sciences (Wiley-Interscience, New York (1993)).

The skilled artisan can construct a mathematical model that predicts clinical response as a function of the level of autoantibodies from the analyses described above. The identification of an association between a clinical response and an expression level for the complexes, autoantibodies or antigens may be the basis for designing a diagnostic method to determine those individuals who will or will not respond to the treatment, or alternatively, will respond at a lower level and thus may require more treatment, i.e., a greater dose of a drug. The only requirement is that there be a good correlation between the diagnostic test results and the underlying condition. In one embodiment, this diagnostic method uses an assay for complexes, antigens and/or autoantibodies described above.

Monitoring Clinical Efficacy.

In one embodiment, the present invention provides for monitoring the influence of treatments (e.g., drugs, compounds, small molecules or devices) on the level of complexes, autoantibodies or antigens. Such assays can also be applied in basic drug screening and in clinical trials. For example, the effectiveness of an agent to increase (or decrease) complex, antigen and/or autoantibody levels can be monitored in clinical trials of subjects. An agent that affects the level of complexes, antigens and/or autoantibodies can be identified by administering the agent and observing a response. In this way, the level of the complexes, antigens and/or autoantibodies can serve as a marker, indicative of the physiological response of the subject to the agent. Accordingly, this response state may be determined before, and at various points during, treatment of the individual with the agent.

Subject Classification.

Standard control levels of complexes, antigens and/or autoantibodies are determined by measuring levels in different control groups. The control levels are then compared with the measured level of complexes, antigens and/or autoantibodies in a given subject. The subject can be classified or assigned to a particular group based on how similar the measured levels were compared to the control levels for a given group.

As one of skill in the art will understand, there will be a certain degree of uncertainty involved in making this determination. Therefore, the standard deviations of the control group levels can be used to make a probabilistic determination and the method of this invention are applicable over a wide range of probability-based group determinations. Thus, for example, and not by way of limitation, in one embodiment, if the measured level of the complexes, antigens and/or autoantibodies falls within 2.5 standard deviations of the mean of any of the control groups, then that individual may be assigned to that group. In another embodiment, if the measured level of the complexes, antigens and/or autoantibodies falls within 2.0 standard deviations of the mean of any of the control groups then that individual may be assigned to that group. In still another embodiment, if the measured level of the complexes, antigens and/or autoantibodies fall within 1.5 standard deviations of the mean of any of the control groups then that individual may be assigned to that group. In yet another embodiment, if the measured level of the complexes, antigens and/or autoantibodies is 1.0 or less standard deviations of the mean of any of the control groups levels then that individual may be assigned to that group. Thus, this process allows determination, with various degrees of probability, which group a specific subject should be placed in, and such assignment would then determine the risk category into which the individual should be placed.

### Substrates

In some embodiments, capture probes may be immobilized on a substrate, i.e., solid support. Examples of such solid supports include plastics such as polycarbonate, complex carbohydrates such as agarose and sepharose, acrylic resins and such as polyacrylamide and latex beads, magnetic beads, and glass slides or glass slides functionalized for attachment of biomolecules. Other examples include SurModic Codelink or Schott Hydrogel slides. Techniques for coupling biomolecules to such solid supports are well known in the art (Weir et al., “Handbook of Experimental Immunology” 4th Ed., Blackwell Scientific Publications, Oxford, England, Chapter 10 (1986); Jacoby et al., Meth. Enzym., 34 Academic Press, N.Y. (1974)).

Appropriate linkers, which can be cross-linking agents, for conjugating a ligand to a solid support include a variety of agents that can react with a functional group present on a surface of the support, or with the ligand, or both. Reagents useful as cross-linking agents include homo-bi-functional and, in particular, hetero-bi-functional reagents. Useful bi-functional cross-linking agents include, but are not limited to, N-SLAB, dimaleimide, DTNB, N-SATA, NSPDP, SMCC and 6-HYNIC. A cross-linking agent can be selected to provide a selectively cleavable bond between a polypeptide and the solid support. For example, a photolabile crosslinker, such as 3-amino-(2-nitrophenyl)propionic acid can be employed as a means for cleaving a polypeptide from a solid support. (Brown et al., Mol. Divers, 4-12 (1995); Rothschild et al., Nucl. Acids Res., 24:351 (1996); and U.S. Pat. No. 5,643,722). Other cross-linking reagents are well-known in the art. (See, e.g., Wong (1991), supra; and Hermanson (1996), supra).

A capture probe, such as a polypeptide can be immobilized on a solid support, such as a coated slide, through a covalent amide bond formed between a carboxyl group functionalized substrate and the amino terminus of the polypeptide or, conversely, through a covalent amide bond formed between an amino group functionalized substrate and the carboxyl terminus of the polypeptide. In addition, a bi-functional trityl linker can be attached to the support, e.g., to the 4-nitrophenyl active ester on a resin, such as a Wang resin, through an amino group or a carboxyl group on the resin via an amino resin. Using a bi-functional trityl approach, the solid support can require treatment with a volatile acid, such as formic acid or trifluoracetic acid to ensure that the polypeptide is cleaved and can be removed. In such a case, the polypeptide can be deposited as a patch at the bottom of a well of a solid support or on the flat surface of a solid support.

Hydrophobic trityl linkers can also be exploited as acid-labile linkers by using a volatile acid or an appropriate matrix solution, e.g., a matrix solution containing 3-HPA, to cleave an amino linked trityl group from the polypeptide. Acid lability can also be changed. For example, trityl, monomethoxytrityl, dimethoxytrityl or trimethoxytrityl can be changed to the appropriate p-substituted, or more acid-labile tritylamine derivatives, of the polypeptide, i.e., trityl ether and tritylamine bonds can be made to the polypeptide. Accordingly, a polypeptide can be removed from a hydrophobic linker, e.g., by disrupting the hydrophobic attraction or by cleaving tritylether or tritylamine bonds under acidic conditions, including, if desired, under typical MS conditions, where a matrix, such as 3-HPA acts as an acid.

A capture probe can be conjugated to a solid support through a noncovalent interaction. For example, a magnetic bead made of a ferromagnetic material, which is capable of being magnetized, can be attracted to a magnetic solid support, and can be released from the support by removal of the magnetic field. Alternatively, the solid support can be provided with an ionic or hydrophobic moiety, which can allow the interaction of an ionic or hydrophobic moiety, respectively, with a polypeptide, e.g., a polypeptide containing an attached trityl group or with a second solid support having hydrophobic character.

A solid support can also be provided with a member of a specific binding pair and, therefore, can be conjugated to a polypeptide containing a complementary binding moiety. For example, a bead coated with avidin or with streptavidin can be bound to a polypeptide having a biotin moiety incorporated therein, or to a second solid support coated with biotin or derivative of biotin, such as imino-biotin. Additionally, a peptide can be covalently conjugated to another carrier protein. The carrier protein could be, for example, Bovine Serum Albumin (BSA), where the coupling takes place using covalent or non-covalent conjugation of the peptide and the carrier protein. The resulting conjugate can be immobilized on a solid support. Alternatively, the carrier protein (e.g., streptavidin or BSA) can be immobilized to a substrate first, followed by immobilization of the peptide.

It should be recognized that any of the binding agents disclosed herein or otherwise known in the art can be reversed. Thus, biotin, e.g., can be incorporated into either a polypeptide or a solid support and, conversely, avidin or other biotin binding moiety would be incorporated into the support or the polypeptide, respectively. Other specific binding pairs contemplated for use herein include, but are not limited to, hormones and their receptors, enzyme, and their substrates, a nucleotide sequence and its complementary sequence, an antibody and the antigen to which it interacts specifically, and other such pairs knows to those skilled in the art.

Any suitable substrate may be used and such substrates may be addressable. A plurality of capture probes (e.g., antigens or antibodies coupled to a carrier molecule), each of which can recognize a different target analyte (e.g., complexes, autoantibodies or antigens), may be attached to the substrate in an array of spots. If desired, each spot of capture probes may be located between two electrodes, the optional label on the detection probe may be a nanoparticle made of a material that is a conductor of electricity, and a change in conductivity may be detected. For example, the electrodes may be made of gold and nanoparticles may be made of gold.

In some embodiments, the methods described herein may detect disease-associated complexes, antigens and/or autoantibodies through a specific binding of a nanoparticle-based detection probe with the complexes, antigens and/or autoantibody. The signal from the nanoparticles may be amplified with a silver or gold enhancement solution from any substrate which allows observation of the detectable change. Suitable substrates include transparent or opaque solid surfaces (e.g., glass, quartz, plastics and other polymers TLC silica plates, filter paper, glass fiber filters, cellulose nitrate membranes, nylon membranes), and conducting solid surfaces (e.g., indium-tin-oxide (ITO, silicon dioxide (SiO₂), silicon oxide (SiO), silicon nitride, etc.)). The substrate can be any shape or thickness, but generally will be flat and thin like a microscope slide or shaped into well chambers like a microtiter plate.

### Detection Probes

In some embodiments, the capture probes bound to the solid support specifically bind to a corresponding molecule to form a complex. Simultaneously or subsequently, the molecule is contacted with a detection probe. In one embodiment, the detection probes are coupled with a label moiety, i.e., detectable group. The particular label or detectable group conjugated to the binding agent is not a critical aspect of the invention, so long as it does not significantly interfere with the specific binding of the binding agent to the target molecule, e.g., human immunoglobulin. In one embodiment, the detection probe comprises a nanoparticle conjugated directly or indirectly to an antibody such as an anti-human Ig antibody, e.g., one or more of an anti-IgG (including autoantibodies that possess Fc domains), anti-IgA, anti-IgM, anti-IgE, and anti-IgD. The nanoparticle-antibody conjugate is contacted with the substrate under conditions effective to allow binding of the target molecule (e.g., autoantibodies) on the substrate with the anti-human Ig antibody.

Nanoparticles useful in the practice of the invention include metal (e.g., gold, silver, copper and platinum), semiconductor (e.g., CdSe, CdS, and CdS or CdSe coated with ZnS) and magnetic (e.g., ferromagnetite) colloidal materials. Other nanoparticles useful in the practice of the invention include ZnS, ZnO, TiO2, AgI, AgBr, HgI2, PbS, PbSe, ZnTe, CdTe, In2S3, In2 Se3, Cd3P2, Cd3As2, InAs, and GaAs. The size of the nanoparticles is preferably from about 5 nm to about 150 nm (mean diameter), more preferably from about 5 to about 50 nm, most preferably from about 10 to about 30 nm. The nanoparticles may also be rods. Other nanoparticles useful in the invention include silica and polymer (e.g., latex) nanoparticles.

Previous studies have demonstrated that biomolecules including DNA and antibodies can be conjugated to gold nanoparticles via a thiol linkage (Mirkin et al., Nature, 382:607 (1996)). The resulting modified gold particles can be used to detect analytes in a variety of formats (See, e.g., Storhoff et al., Chem. Rev., 99:1849 (1999); Niemeyer, C. M. Angew. Chem. Int. Ed., 40:4128 (2001); Liu et al., J. Am. Chem. Soc., 125:6642 (2003)), including DNA microarrays, where high detection sensitivity is achieved in conjunction with silver amplification (Taton et al., Science, 289:1757 (2000); Storhoff et al., Biosens. Bioelectron, 19:875 (2004)).

An effective method for functionalizing nanoparticles with biomolecules has been developed. See U.S. Pat. Nos. 6,361,944 and 6,417,340 (Nanosphere, Inc.), which are incorporated by reference in their entirety. The process leads to nanoparticles that are heavily functionalized and have enhanced particle stability. The resulting modified particles have also proven to be very robust as evidenced by their stability in solutions containing elevated electrolyte concentrations, stability towards centrifugation or freezing, and thermal stability when repeatedly heated and cooled. This loading process also is controllable and adaptable. Such methods can also be used to generate nanoparticle-antibody or nanoparticle-biotin conjugates.

In other embodiments, the detectable group can be any material having a detectable physical or chemical property. Such detectable labels have been well-developed in the field of immunoassays and imaging, in general, most any label useful in such methods can be applied to the present invention. Useful labels include magnetic beads (e.g., Dynabeads™), fluorescent dyes (e.g., fluorescein isothiocyanate, Texas red, rhodamine, and the like), radiolabels (e.g., 3H, 14C, 35S, 125I, 121I, 131I, 112In, 99 mTc), other imaging agents such as microbubbles (for ultrasound imaging), 18F, 11C, 15O, (for Positron emission tomography), 99 mTC, 111In (for Single photon emission tomography), enzymes (e.g., horse radish peroxidase, alkaline phosphatase and others commonly used in an ELISA), and calorimetric labels such as colloidal gold or colored glass or plastic (e.g., polystyrene, polypropylene, latex, and the like) beads. Patents that described the use of such labels include U.S. Pat. Nos. 3,817,837; 3,850,752; 3,939,350; 3,996,345; 4,277,437; 4,275,149; and 4,366,241, each incorporated herein by reference in their entirety and for all purposes. See also Handbook of Fluorescent Probes and Research Chemicals (6th Ed., Molecular Probes, Inc., Eugene Oreg.).

The nanoparticle may be linked to an antibody either directly or indirectly. For example, the nanoparticle may be directly functionalized with the desired detection antibody. Alternatively, the nanoparticle may be functionalized with a biotin moiety and the desired detection antibody is also functionalized with a biotin moiety. An avidin or streptavidin molecule is used to link (i.e., “bridge”) the nanoparticle to the antibody. The antibody nanoparticle conjugate may be formed by step-wise addition of the antibody, streptavidin, and biotinylated nanoparticle to the substrate. For example, see U.S. Provisional Application Ser. No. 61/036,892 filed on Mar. 14, 2008, which is hereby incorporated by reference herein in its entirety and U.S. Provisional Application Ser. No. 61/055,875 filed on May 23, 2008, which is hereby incorporated by reference herein in its entirety. Receptor-ligand pairs alternative to streptavidin-biotin also may be used. For instance, the FITC anti-FITC system is a well known alternative to biotin streptavidin. Additionally, double-headed protease inhibitors (Black-eyed pea chymotrypsin or trypsin inhibitor) bind two molecules of protease simultaneously (Gennis et al., J. Biol. Chem., 251:741). As such, the inhibitors can be used to link the nanoparticle and the antibody using two connecting genetically modified proteases.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidoreductases, particularly peroxidases. Fluorescent compounds useful as labelling moieties, include, but are not limited to, e.g., fluorescein and its derivatives, rhodamine and its derivatives, dansyl, umbelliferone, and the like. Chemiluminescent compounds useful as labelling moieties, include, but are not limited to, e.g., luciferin, and 2,3-dihydrophthalazinediones, e.g., luminol. For a review of various labeling or signal-producing systems which can be used, see, U.S. Pat. No. 4,391,904.

### Detection

Means of detecting labels are well known to those of skill in the art. Thus, for example, where the label is a radioactive label, means for detection include a scintillation counter or photographic film for autoradiography. Where the label is a fluorescent label, it can be detected by exciting the fluorochrome with the appropriate wavelength of light and detecting the resulting fluorescence. The fluorescence can be detected visually, by means of photographic film, by the use of electronic detectors such as charge coupled devices (CCDs) or photomultipliers and the like. Similarly, enzymatic labels can be detected by providing the appropriate substrates for the enzyme and detecting the resulting reaction product. Finally simple colorimetric labels can be detected simply by observing the color associated with the label.

In some embodiments, a colorimetric method for monitoring scattered light may be used to detect the nanoparticle conjugates. See U.S. Ser. No. 10/995,051, filed Nov. 22, 2004, which is incorporated by reference in its entirety. Moreover, the methods enable the detection of probe-target complexes containing two or more particles in the presence of a significant excess of non-complexed particles, which drives hybridization in the presence of low target concentrations.

Nanoparticle detection probes, particularly gold nanoparticle probes conjugated to antibodies, or those conjugated to ligands for another molecule, e.g., nanoparticles conjugated to streptavidin, are suitable for detection of complexes, antigens and/or autoantibodies. A silver-based signal amplification procedure can further provide ultra-high sensitivity enhancement. Silver staining can be employed with any type of nanoparticles that catalyze the reduction of silver and can be used to produce or enhance a detectable change in any assay performed on a substrate, including those described above.

A nanoparticle can also be detected, for example, using resonance light scattering, after illumination by various methods including dark-field microscopy, evanescent waveguides, or planar illumination of glass substrates. Metal particles >40 nm diameter scatter light of a specific color at the surface plasmon resonance frequency (Yguerabide et al., Anal. Biochem., 262:157 (1998)), and can be used for multicolor labeling on substrates by controlling particle size, shape, and chemical composition (Taton et al., J. Am. Chem. Soc., 123:5164 (2001); Jin et al., Science, 294:1901 (2001)). In another embodiment, a nanoparticle can be detected in a method of the invention, for example, using surface enhanced raman spectroscopy (SERS) in either a homogeneous solution based on nanoparticle aggregation (Graham et al., Angew. Chem., 112:1103 (2000)), or on substrates in a solid-phase assay (Porter et al., Anal. Chem., 71:4903 (1999)), or using silver development followed by SERS (Mirkin et al., Science, 297:1536 (2002)). In another embodiment, the nanoparticles may be detected by photothermal imaging (Boyer et al., Science, 297:1160 (2002)), diffraction based sensing technology (Bailey et. al, J. Am. Chem. Soc., 125:13541 (2003)), or hyper-Rayleigh scattering (Kim et al., Chem. Phys. Lett., 352:421 (2002)).

A nanoparticle can be detected in a method of the invention, for example, using an optical or flatbed scanner. The scanner can be linked to a computer loaded with software capable of calculating grayscale measurements, and the grayscale measurements are calculated to provide a quantitative measure of the amount of analyte detected. Suitable scanners include those used to scan documents into a computer which are capable of operating in the reflective mode (e.g., a flatbed scanner), other devices capable of performing this function or which utilize the same type of optics, any type of grayscale-sensitive measurement device, and standard scanners which have been modified to scan substrates according to the invention. The software can also provide a color number for colored spots and can generate images (e.g., printouts) of the scans, which can be reviewed to provide a qualitative determination of the presence of a nucleic acid, the quantity of a nucleic acid, or both. In addition, it has been found that the sensitivity of assays can be increased by subtracting the color that represents a negative result from the color that represents a positive result.

### Nanoparticles

In general, nanoparticles (NPs) contemplated include any compound or substance, including for example and without limitation, a metal, a semiconductor, and an insulator particle composition, and a dendrimer (organic or inorganic). The term “functionalized nanoparticle,” as used herein, refers to a nanoparticle having at least a portion of its surface modified with a distinct molecule.

Thus, nanoparticles are contemplated for use in the methods which comprise a variety of inorganic materials including, but not limited to, metals, semi-conductor materials or ceramics as described in U.S. Patent Publication No 20030147966. For example, metal-based nanoparticles include those described herein. Ceramic nanoparticle materials include, but are not limited to, brushite, tricalcium phosphate, alumina, silica, and zirconia. Organic materials from which nanoparticles are produced include carbon. Nanoparticle polymers include polystyrene, silicone rubber, polycarbonate, polyurethanes, polypropylenes, polymethylmethacrylate, polyvinyl chloride, polyesters, polyethers, and polyethylene. Biodegradable, biopolymer (e.g. polypeptides such as BSA, polysaccharides, etc.), other biological materials (e.g. carbohydrates), and/or polymeric compounds are also contemplated for use in producing nanoparticles.

In one embodiment, the nanoparticle is metallic, and in various aspects, the nanoparticle is a colloidal metal. Thus, in various embodiments, nanoparticles useful in the practice of the methods include metal (including for example and without limitation, gold, silver, platinum, aluminum, palladium, copper, cobalt, indium, nickel, or any other metal amenable to nanoparticle formation), semiconductor (including for example and without limitation, CdSe, CdS, and CdS or CdSe coated with ZnS) and magnetic (for example, ferromagnetite) colloidal materials, as well as silica containing materials. Other nanoparticles useful in the practice of the invention include, also without limitation, ZnS, ZnO, Ti, TiO₂, Sn, SnO₂, Si, SiO₂, Fe, Fe⁺⁴, Ag, Cu, Ni, Al, steel, cobalt-chrome alloys, Cd, titanium alloys, AgI, AgBr, HgI₂, PbS, PbSe, ZnTe, CdTe, In₂S₃, In₂Se₃, Cd₃P₂, Cd₃As₂, InAs, and GaAs. The size of the nanoparticles may be from about 5 nm to about 150 nm (mean diameter), e.g., from about 5 to about 50 nm, or from about 10 to about 30 nm. The nanoparticles may also be rods. Methods of making ZnS, ZnO, TiO₂, AgI, AgBr, HgI₂, PbS, PbSe, ZnTe, CdTe, In₂S₃, In₂Se₃, Cd₃P₂, Cd₃As₂, InAs, and GaAs nanoparticles are also known in the art. See, e.g., Weller, Angew. Chem. Int. Ed. Engl., 32:41 (1993); Henglein, Top. Curr. Chem., 143:113 (1988); Henglein, Chem. Rev., 89:1861 (1989); Brus, Appl. Phys. A., 53:465 (1991); Bahncmann, in Photochemical Conversion and Storage of Solar Energy (eds. Pelizetti and Schiavello 1991), page 251; Wang and Herron, J. Phys. Chem., 95:525 (1991); Olshaysky, et al., J. Am. Chem. Soc., 112:9438 (1990); Ushida et al., J. Phys. Chem., 95, 5382 (1992).

In practice, methods are provided using any suitable nanoparticle having a distinct molecule attached thereto, e.g., streptavidin or an antibody, that are in general suitable for use in detection assays known in the art to the extent and do not interfere with complex formation The size, shape and chemical composition of the particles contribute to the properties of the resulting functionalized nanoparticle. These properties include for example, optical properties, optoelectronic properties, electrochemical properties, electronic properties, stability in various solutions, magnetic properties, and pore and channel size variation. The use of mixtures of particles having different sizes, shapes and/or chemical compositions, as well as the use of nanoparticles having uniform sizes, shapes and chemical composition, is contemplated. Examples of suitable particles include, without limitation, nanoparticles, aggregate particles, isotropic (such as spherical particles) and anisotropic particles (such as non-spherical rods, tetrahedral, prisms) and core-shell particles such as the ones described in U.S. Pat. No. 7,238,472 and International Patent Publication No. WO 2002/096262, the disclosures of which are incorporated by reference in their entirety.

Methods of making metal, semiconductor and magnetic nanoparticles are well-known in the art. See, for example, Schmid, G. (ed.) Clusters and Colloids (VCH, Weinheim, 1994); Hayat, M. A. (ed.) Colloidal Gold: Principles, Methods, and Applications (Academic Press, San Diego, 1991); Massart, R., IEEE Transactions On Magnetics, 17, 1247 (1981); Ahmadi, T. S. et al., Science, 272, 1924 (1996); Henglein, A. et al., J. Phys. Chem., 99, 14129 (1995); Curtis, A. C., et al., Angew. Chem. Int. Ed. Engl., 27, 1530 (1988). Preparation of polyalkylcyanoacrylate nanoparticles prepared is described in Fattal, et al., J. Controlled Release (1998) 53: 137-143 and U.S. Pat. No. 4,489,055. Methods for making nanoparticles comprising poly(D-glucaramidoamine)s are described in Liu, et al., J. Am. Chem. Soc. (2004) 126:7422-7423. Preparation of nanoparticles comprising polymerized methylmethacrylate (MMA) is described in Tondelli, et al., Nucl. Acids Res. (1998) 26:5425-5431, and preparation of dendrimer nanoparticles is described in, for example Kukowska-Latallo, et al., Proc. Natl. Acad. Sci. USA (1996) 93:4897-4902 (Starburst polyamidoamine dendrimers).

Suitable nanoparticles are also commercially available from, for example, Ted Pella, Inc. (gold), Amersham Corporation (gold) and Nanoprobes, Inc. (gold).

Also as described in U.S. Patent Publication No. 20030147966, nanoparticles comprising materials described herein are available commercially or they can be produced from progressive nucleation in solution (e.g., by colloid reaction), or by various physical and chemical vapor deposition processes, such as sputter deposition. See, e.g., HaVashi, (1987) Vac. Sci. Technol. July/August 1987, A5(4):1375-84; Hayashi, (1987) Physics Today, December 1987, pp. 44-60; MRS Bulletin, January 1990, pp. 16-47.

As further described in U.S. Patent Publication No. 20030147966, nanoparticles contemplated are produced using HAuCl₄ and a citrate-reducing agent, using methods known in the art. See, e.g., Marinakos et al., (1999) Adv. Mater. 11: 34-37; Marinakos et al., (1998) Chem. Mater. 10: 1214-19; Enustun & Turkevich, (1963) J. Am. Chem. Soc. 85: 3317. Tin oxide nanoparticles having a dispersed aggregate particle size of about 140 nm are available commercially from Vacuum Metallurgical Co., Ltd. of Chiba, Japan. Other commercially available nanoparticles of various compositions and size ranges are available, for example, from Vector Laboratories, Inc. of Burlingame, Calif.

### Nanoparticle Size

In various aspects, methods provided include those utilizing nanoparticles which range in size from about 1 nm to about 250 nm in mean diameter, about 1 nm to about 240 nm in mean diameter, about 1 nm to about 230 nm in mean diameter, about 1 nm to about 220 nm in mean diameter, about 1 nm to about 210 nm in mean diameter, about 1 nm to about 200 nm in mean diameter, about 1 nm to about 190 nm in mean diameter, about 1 nm to about 180 nm in mean diameter, about 1 nm to about 170 nm in mean diameter, about 1 nm to about 160 nm in mean diameter, about 1 nm to about 150 nm in mean diameter, about 1 nm to about 140 nm in mean diameter, about 1 nm to about 130 nm in mean diameter, about 1 nm to about 120 nm in mean diameter, about 1 nm to about 110 nm in mean diameter, about 1 nm to about 100 nm in mean diameter, about 1 nm to about 90 nm in mean diameter, about 1 nm to about 80 nm in mean diameter, about 1 nm to about 70 nm in mean diameter, about 1 nm to about 60 nm in mean diameter, about 1 nm to about 50 nm in mean diameter, about 1 nm to about 40 nm in mean diameter, about 1 nm to about 30 nm in mean diameter, or about 1 nm to about 20 nm in mean diameter, about 1 nm to about 10 nm in mean diameter. In other aspects, the size of the nanoparticles is from about 5 nm to about 150 nm (mean diameter), from about 5 to about 50 nm, from about 10 to about 30 nm. The size of the nanoparticles is from about 5 nm to about 150 nm (mean diameter), from about 30 to about 100 nm, from about 40 to about 80 nm. The size of the nanoparticles used in a method varies as required by their particular use or application. The variation of size is advantageously used to optimize certain physical characteristics of the nanoparticles, for example, optical properties or amount surface area that can be derivatized as described herein.

### Exemplary Solid Substrates

Any substrate which allows observation of a detectable change, e.g., an optical change, may be employed in the methods of the invention. Suitable substrates include transparent solid surfaces (e.g., glass, quartz, plastics and other polymers), opaque solid surface (e.g., white solid surfaces, such as TLC silica plates, filter paper, glass fiber filters, cellulose nitrate membranes, nylon membranes), and conducting solid surfaces (e.g., indium-tin-oxide (ITO), silicon dioxide (SiO₂), silicon oxide (SiO), silicon nitride, etc.)). The substrate can be any shape or thickness, but generally is flat and thin. In one embodiment, the substrates are transparent substrates such as glass (e.g., glass slides) or plastics (e.g., wells of microtiter plates).

### Antibody Based Assays

Proteins such as Tau, or aggregates thereof, Abeta, ADDLs, globulomers, variants thereof or fragments thereof, may be contacted with a panel of moieties such as aptamers or antibodies or fragments or derivatives thereof specific for the protein. The antibodies or other binding molecules may be affixed to a solid support such as a chip. Binding of proteins indicative of a particular epitope or isoform of Tau, or aggregates thereof, Abeta, ADDLs, globulomers, variants thereof or fragments thereof, may be verified by binding to a detectably labelled secondary antibody or aptamer. For the labelling of antibodies, it is referred to Harlow and Lane, “Antibodies, A Laboratory Manual”, CSH Press, 1988, Cold Spring Harbor. For instance, antibodies against the proteins are immobilized on a solid substrate, e.g., glass slides or microtiter plates. The immobilized complexes can be labeled with a reagent specific for the protein(s). The reactants can include enzyme substrates, DNA, receptors, antigens or antibodies to provide, for example, a capture sandwich immunoassay.

Any of a variety of known immunoassay methods can be used for detection, including, but not limited to, immunoassay, using an antibody specific for the encoded polypeptide, immunoprecipitation, an enzyme immunoassay, e.g., by enzyme-linked immunosorbent assay (ELISA), radioimmunoassay (RIA), and the like.

Given that immunoassay sensitivity is defined not only by the detection system but by the binding affinities of the antibodies involved, it is possible for other detection methods used in commercially available technologies and also those previously defined in the academic literature but not commercially available to reach the assay sensitivities described in the present specification through the use of antibodies with particular binding affinities, or improvements to the detection method or assay methdology. Any of a variety of known immunoassay methods can be used for detection, including, but not limited to, immunoassay, using an antibody specific for the encoded polypeptide, e.g., by enzyme-linked immunosorbent assay (ELISA), radioimmunoassay (RIA), rolling circe amplification (RCA), immunoPCR (iPCR), magnetic bead based assays that utilize fluorescence and chemiluminescence, electrochemiluminescence and the like; and functional assays for the encoded polypeptide, e.g., binding activity or enzymatic activity.

As will be readily apparent to the ordinarily skilled artisan upon reading the present specification, the detection methods and other methods described herein can be varied. Such variations are within the intended scope of the invention. For example, in the above detection scheme, the probe for use in detection can be immobilized on a solid support, and the test sample contacted with the immobilized probe. Binding of the test sample to the probe can then be detected in a variety of ways, e.g., by detecting a detectable label bound to the test sample.

The methods generally include contacting the sample with a detection antibody specific for one or more of Tau, or aggregates thereof, Abeta, ADDLs, globulomers, variants thereof or fragments thereof, or complexes thereof, bound to a capture probe on a solid substrate and detecting binding between the detection antibody and Tau, or aggregates thereof, Abeta, ADDLs, globulomers, variants thereof or fragments thereof, or complexes thereof, in the sample. The level of antibody binding indicates the susceptibility (at risk for, propensity or affirmative diagnosis) of the patient for neurological disorders including but not limited to MLD and Alzheimer's disease. Suitable controls include a sample known not to contain Tau, Abeta, addls or globulimers; a sample contacted with an antibody not specific for Tau, Abeta, addls or globulimers; a sample having a level of Tau, Abeta, addls or globulimers associated with neurological disorders including but not limited to MLD and Alzheimer's disease, or any combination thereof.

In one embodiment, the methods include contacting the sample with a detection antibody specific for Tau, or aggregates thereof, Abeta, ADDLs, globulomers, variants thereof or fragments thereof, and detecting binding between the antibody and molecules of the sample. The level of antibody binding (either qualitative or quantitative) may indicate the susceptibility of the patient to a disease. For example, where the marker polypeptide is present at a level greater than that associated with a negative control level, then the patient is susceptable to disease.

In general, one of the binding moieties, e.g., antibody, is detectably labeled, either directly or indirectly. Direct labels include radioisotopes; enzymes having detectable products (e.g., luciferase, β-galactosidase, and the like); fluorescent labels (e.g., fluorescein isothiocyanate, rhodamine, phycoerythrin, and the like); fluorescence emitting metals, e.g., ¹⁵²Eu, or others of the lanthanide series, attached to the antibody through metal chelating groups such as EDTA; chemiluminescent compounds, e.g., luminol, isoluminol, acridinium salts, and the like; bioluminescent compounds, e.g., luciferin, aequorin (green fluorescent protein), and the like. Indirect labels include members of specific binding pairs, e.g., biotin-avidin, and the like.

One of the binding moieties, e.g., antibody, may be attached (coupled) to an insoluble support, such as a polystyrene plate or a bead. In one embodiment, the sample may be brought into contact with the immobilized antibody and the support washed with suitable buffers followed by contact with a detectably labeled specific antibody. In one embodiment, the sample may be brought into contact with and immobilized on a solid support or carrier, such as nitrocellulose, that is capable of immobilizing soluble proteins. The support may then be washed with suitable buffers followed by contacting with an optionally detectably labeled first specific antibody. Detection methods are known in the art and are chosen as appropriate to the signal emitted by the detectable label. Detection is generally accomplished in comparison to suitable controls, and to appropriate standards.

In one embodiment, the antibody may be attached (coupled) to an insoluble support, such as a polystyrene plate or a bead. Indirect labels include second antibodies specific for antibodies specific for the encoded polypeptide (“first specific antibody”), wherein the second antibody is labeled as described above; and members of specific binding pairs, e.g., biotin-avidin, and the like. The biological sample may be brought into contact with and immobilized on a solid support or carrier, such as nitrocellulose, that is capable of immobilizing cells, cell particles, or soluble proteins. The support may then be washed with suitable buffers, followed by contacting with a detectably-labeled first specific antibody. Detection methods are known in the art and will be chosen as appropriate to the signal emitted by the detectable label. Detection is generally accomplished in comparison to suitable controls, and to appropriate standards.

Polypeptide arrays provide a high throughput technique that can assay a large number of polypeptides in a sample. This technology can be used as a tool to test for presence of a marker polypeptide and assessment of disease. Of particular interest are arrays which comprise a probe for detection of one or more of the marker polypeptides of interest.

A variety of methods of producing arrays of binding molecules, as well as variations of these methods, are known in the art and contemplated for use in the invention. For example, arrays can be created by spotting binding moieties onto a substrate (e.g., glass, nitrocellulose, and the like) in a two-dimensional matrix or array having bound probes. Arrays also can be created by spotting polypeptide probes onto a substrate in a three-dimensional matrix (e.g. hydrogel) or array having bound probes. The probes can be bound to the substrate by either covalent bonds or by non-specific interactions, such as hydrophobic interactions.

Samples of Tau, or aggregates thereof, Abeta, ADDLs, globulomers, variants thereof or fragments thereof, can be detectably labeled (e.g., using radioactive or fluorescent labels) and then contacted with the binding moieties.

Alternatively, the test sample can be immobilized on the array, and the binding moieties detectably labeled and then applied to the immobilized polypeptides. In one embodiment, a binding moiety is detectably labeled. In other embodiments, the binding moiety is immobilized on the array and not detectably labeled. In such embodiments, the sample is applied to the array and bound molecules are detected using labeled binding moieties. In one embodiment, the secondary label probes can be introduced in a direct sandwich format where a primary antibody is bound to the substrate, and the secondary antibody is directly attached to the label such as a gold nanoparticle, which “sandwiches” the target protein when both the primary and secondary antibody binds to epitopes of the target. An alternative methodology well known in the art is to use a secondary antibody in an indirect sandwich assay where the antibody is label with a hapten such as biotin, which can then recognize a streptavidin or avidin molecule which is directly labeled or indirectly labeled.

Other methods well known in the art are competitive immunoassay formats where the signal the presence of known amount of target added to the sample competes against an unknown amount of target present in the sample.

Examples of such protein arrays are described in the following patents or published patent applications: U.S. Pat. No. 6,225,047; PCT International Publication No. WO 99/51773; U.S. Pat. No. 6,329,209; PCT International Publication No. WO 00/56934; and U.S. Pat. No. 5,242,828.

### Algorithms and Computer Applications

The invention also provides a variety of computer-related embodiments. Specifically, the automated means for performing the methods described above may be controlled using computer-readable instructions, i.e., programming. Accordingly, in some embodiments the invention provides computer programming for analyzing and comparing protein patterns present in a sample, wherein the comparing indicates the presence or absence of a disease.

In another embodiment, the invention provides computer programming for analyzing and comparing protein patterns from samples taken from a subject, e.g., at at least two different time points or different proteins, wherein the pattern is indicative of a disease. In one embodiment, the comparing provides for monitoring of the progression of the disease from the first time point to the second time point.

The methods and systems described herein can be implemented in numerous ways. In one embodiment of particular interest, the methods involve use of a communications infrastructure, for example the internet. Several embodiments of the invention are discussed below. It is also to be understood that the present invention may be implemented in various forms of hardware, software, firmware, processors, or a combination thereof. The methods and systems described herein can be implemented as a combination of hardware and software. The software can be implemented as an application program tangibly embodied on a program storage device, or different portions of the software implemented in the user's computing environment (e.g., as an applet) and on the reviewer's computing environment, where the reviewer may be located at a remote site (e.g., at a service provider's facility).

For example, during or after data input by the user, portions of the data processing can be performed in the user-side computing environment. For example, the user-side computing environment can be programmed to provide for defined test codes to denote platform, carrier/diagnostic test, or both; processing of data using defined flags, and/or generation of flag configurations, where the responses are transmitted as processed or partially processed responses to the reviewer's computing environment in the form of test code and flag configurations for subsequent execution of one or more algorithms to provide a results and/or generate a report in the reviewer's computing environment.

The application program for executing the algorithms described herein may be uploaded to, and executed by, a machine comprising any suitable architecture. In general, the machine involves a computer platform having hardware such as one or more central processing units (CPU), a random access memory (RAM), and input/output (I/O) interface(s). The computer platform also includes an operating system and microinstruction code. The various processes and functions described herein may either be part of the microinstruction code or part of the application program (or a combination thereof) which is executed via the operating system. In addition, various other peripheral devices may be connected to the computer platform such as an additional data storage device and a printing device.

As a computer system, the system generally includes a processor unit. The processor unit operates to receive information, which generally includes test data (e.g., protein levels or patterns tested), and test result data (e.g., the levels of specific proteins within a sample). This information received can be stored at least temporarily in a database, and data analyzed in comparison to a library of known protein patterns to be indicative of the presence or absence of a disease.

Part or all of the input and output data can also be sent electronically; certain output data (e.g., reports) can be sent electronically or telephonically (e.g., by facsimile, e.g., using devices such as fax back). Exemplary output receiving devices can include a display element, a printer, a facsimile device and the like. Electronic forms of transmission and/or display can include email, interactive television, and the like. In an embodiment of particular interest, all or a portion of the input data and/or all or a portion of the output data (e.g., usually at least the protein levels known to be indicative of the presence or absence of a disease) are maintained on a server for access, preferably confidential access. The results may be accessed or sent to professionals as desired.

A system for use in the methods described herein generally includes at least one computer processor (e.g., where the method is carried out in its entirety at a single site) or at least two networked computer processors (e.g., where protein pattern data for a sample obtained from a subject is to be input by a user (e.g., a technician or someone performing the activity assays)) and transmitted to a remote site to a second computer processor for analysis (e.g., where the protein pattern data is compared to a library of protein patterns known to be indicative of the presence or absence of a disease), where the first and second computer processors are connected by a network, e.g., via an intranet or internet). The system can also include a user component(s) for input; and a reviewer component(s) for review of data, and generation of reports, including detection of disease, differential diagnosis or monitoring the progression of a disease. Additional components of the system can include a server component(s); and a database(s) for storing data (e.g., as in a database of report elements, e.g., a library of protein patterns known to be indicative of the presence or absence of a disease, or a relational database (RDB) which can include data input by the user and data output. The computer processors can be processors that are typically found in personal desktop computers (e.g., IBM, Dell, Macintosh), portable computers, mainframes, minicomputers, or other computing devices.

The networked client/server architecture can be selected as desired, and can be, for example, a classic two or three tier client server model. A relational database management system (RDMS) either as part of an application server component or as a separate component (RDB machine) provides the interface to the database.

In one embodiment, the architecture is provided as a database-centric user/server architecture, in which the user application generally requests services from the application server which makes requests to the database (or the database server) to populate the activity assay report with the various report elements as required, especially the assay results for each activity assay. The server(s) (e.g., either as part of the application server machine or a separate RDB/relational database machine) responds to the user's requests.

The input components can be complete, stand-alone personal computers offering a full range of power and features to run applications. The user component usually operates under any desired operating system and includes a communication element (e.g., a modem or other hardware for connecting to a network), one or more input devices (e.g., a keyboard, mouse, keypad, or other device used to transfer information or commands), a storage element (e.g., a hard drive or other computer-readable, computer-writable storage medium), and a display element (e.g., a monitor, television, LCD, LED, or other display device that conveys information to the user). The user enters input commands into the computer processor through an input device. Generally, the user interface is a graphical user interface (GUI) written for web browser applications.

The server component(s) can be a personal computer, a minicomputer, or a mainframe and offers data management, information sharing between clients, network administration and security. The application and any databases used can be on the same or different servers.

Other computing arrangements for the user and server(s), including processing on a single machine such as a mainframe, a collection of machines, or other suitable configuration are contemplated. In general, the user and server machines work together to accomplish the processing of the present invention.

Where used, the database(s) is usually connected to the database server component and can be any device which will hold data. For example, the database can be any magnetic or optical storing device for a computer (e.g., CDROM, internal hard drive, tape drive). The database can be located remote to the server component (with access via a network, modem, etc.) or locally to the server component.

Where used in the system and methods, the database can be a relational database that is organized and accessed according to relationships between data items. The relational database is generally composed of a plurality of tables (entities). The rows of a table represent records (collections of information about separate items) and the columns represent fields (particular attributes of a record). In its simplest conception, the relational database is a collection of data entries that “relate” to each other through at least one common field.

Additional workstations equipped with computers and printers may be used at point of service to enter data and, in some embodiments, generate appropriate reports, if desired. The computer(s) can have a shortcut (e.g., on the desktop) to launch the application to facilitate initiation of data entry, transmission, analysis, report receipt, etc. as desired.

### Kits

Also within the scope of the disclosure are kits comprising capture and detection probe compositions and instructions for use. The kits are useful for detecting the presence of autoantibodies to Tau, or aggregates thereof, Abeta, ADDLs, globulomers, variants thereof or fragments thereof; presence of Tau, or the presence of Tau or aggregates thereof, Abeta, ADDLs, globulomers, variants thereof or fragments thereof, or complexes thereof, in a biological sample, e.g., any body fluid including, but not limited to, blood or components thereof, blood or components thereof, lymph, cystic fluid, urine, stool, cerebrospinal fluid, acitic fluid or blood or components thereof and including biopsy samples of body tissue. For example, the kit can comprise: one or more capture probes and/or detection probes; means for determining the amount of the autoantibodies or Tau, or aggregates thereof, Abeta, ADDLs, globulomers, variants thereof or fragments thereof, or complexes thereof, in the sample; and means for comparing the amount of the autoantibodies or Tau, or aggregates thereof, Abeta, ADDLs, globulomers, variants thereof or fragments thereof, or complexes thereof, in the sample with a standard. One or more of the detection probes may be labeled. The kit components, (e.g., reagents) can be packaged in a suitable container. The kit can further comprise instructions for using the kit to detect the autoantibodies or Tau, or aggregates thereof, Abeta, ADDLs, globulomers, variants thereof or fragments thereof, or complexes thereof.

In one embodiment, the kit includes: (1) a capture probe (e.g., as described herein above); and (2) a detection probe which may be an antibody which binds to the analyte as described above and is conjugated (directly or indirectly) to a nanoparticle. The kit can also include, e.g., a buffering agent, a preservative or a protein-stabilizing agent. The kit can further include components necessary for detecting the detectable-label, e.g., an enzyme or a substrate. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits may contain a written product on or in the kit container. The written product describes how to use the reagents contained in the kit, e.g., to use the autoantibodies, complexes or antigen in determining a strategy for preventing or treating neurological disorders including but not limited to MLD and Alzheimer's disease in a subject. In several embodiments, the use of the reagents can be according to the methods described herein.

The invention will be further described by the following nonlimiting examples.

### EXAMPLES

### Example 1

### Preparation of Gold Nanoparticles

Previous studies have demonstrated that biomolecules including DNA and antibodies can be conjugated to gold nanoparticles via a thiol linkage (Mirkin et al., Nature 382:607-609 (1996)). The resulting modified gold particles have been used to detect analytes in a variety of formats (See, e.g., Storhoff et al., Chem. Rev., 99:1849-1862 (1999); Niemeyer, C. M. Angew. Chem. Int. Ed., 40:4128-4158 (2001); Liu et al., J. Am. Chem. Soc., 125:6642-6643 (2003)), including DNA microarrays, where high detection sensitivity is achieved in conjunction with silver amplification (Taton et al., Science, 289:1757-1760 (2000); Storhoff et al., Biosens. Bioelectron, 19:875-883 (2004)). Additional key features of this technology include the remarkable stability and robustness of the modified gold nanoparticles which withstand both elevated temperatures and salt concentrations (Mirkin et al. Nature, 382:607-609 (1996); Storhoff et al., Langmuir, 18:6666-6670 (2002)), as well as the remarkable specificity by which target analytes are recognized (Storhoff et al., J. Am. Chem. Soc., 120:1959-1964 (1998); Taton et al., Am. Chem. Soc., 122:6305-6306 (2000)).

Gold colloids (about 15 nm diameter) are prepared by reduction of HAuC14 with citrate as described in Frens, Nature Phys. Sci., 241:20-22 (1973) and Grabar, Anal. Chem., 67:735 (1995). Briefly, all glassware is cleaned in aqua regia (3 parts HCl, 1 part HNO₃), rinsed with Nanopure H2O, then oven dried prior to use. HAuC14 and sodium citrate are purchased from Aldrich Chemical Company. Aqueous HAuC14 (1 mM, 500 mL) is brought to reflux while stirring. Then, 38.8 mM sodium citrate (50 mL) is added quickly. The solution color changed from pale yellow to burgundy, and refluxing is continued for 15 min. After cooling to room temperature, the red solution is filtered through a Micron Separations Inc. 0.2 micron cellulose 33 acetate filter. Au colloids are characterized by UV-vis spectroscopy using a Hewlett Packard 8452A diode array spectrophotometer and by Transmission Electron Microscopy (TEM) using a Hitach±8100 transmission electron microscope.

### Example 2

### Preparation of Probe-Coated Substrates

Purified capture probe (e.g., any one or more of antibodies that bind Tau, or aggregates thereof, Abeta, ADDLs, globulomers, variants thereof or fragments thereof, or complexes thereof, one or more Tau, Abeta, addls or globulimers, or autoantibodies that bind Tau, Abeta, addls or globulimers in neurological disorders including but not limited to MLD and Alzheimer's disease subjects) are synthesized according to standard procedures. The antibodies, proteins or peptides are arrayed onto Codelink (Amersham, Inc.) or Hydrogel substrates (Nexterion Slide H Hydrogel Coated Substrate) using a GMS417 arrayer (Affymetrix). The substrates are incubated overnight in a humidity chamber, and subsequently washed with TBS-T Buffer (150 mM NaCl/10 mM Tris Base buffer (pH 8) containing 0.05% Tween. All of the proteins are arrayed in triplicate. The position of the arrayed spots is designed to allow multiple assays on each substrate, achieved by partitioning the substrate into separate test wells by silicon gaskets (Grace Biolabs). For example, the following capture probes are arrayed on a slide:

Sample Capture Probe Water (μL) 4× Printing Buffer (μL)

1 100 μL of BSA (80 ng/μL) 50 50

2 50 μL of BSA (80 ng/μL) 100 50

3 25 μL of BSA (80 ng/μL) 125 50

4 5 μL of BSA (80 ng/μL) 145 50

5 30 μL peptide antigen (80 ng/μL) 60 30

6 30 μL peptide antigen (80 ng/μL) 87 3

7 60 μL peptide antigen (80 ng/μL) 30 30

8 60 μL peptide antigen (80 ng/μL) 57 3

9 85 μL of capture probe (100 ng/μL) 74 53

106 μL sample 9 106

11 106 μL sample 10 106

12 40 μL sample 11 160

Following binding, the slides are rinsed two times with 1×PBS/0.3% Tween (200 μL. The slides are then incubated with blocking solution (25 mM NaCl/25 mM Tris, pH 8.0/25 mM ethanolamine/0.15% Tween 20/0.5×PBS/0.5% BSA) for Codelink and Hydrogel slides at room temperature (23° C.), 250 rpm for 60 min. Finally, the slides are rinsed two times with 150 mM NaNO3/0.3% Tween.

### Example 3

### Detection of Autoantibodies

In an illustrative embodiment, test samples are assayed as follows. One hundred microliters (100 μL) of the samples (1% blood or components thereof sample dilution) are added to each well and incubated at room temperature, with shaking at 250 rpm for 10 min. Next, the target binding solution is shaken off and the plate is washed three times with 150 mM NaNO3/0.3% TW. A biotinantibody mixture (100 μL of 50 ng/100 μL in binding buffer) is added to each well and the slides are incubated at 23° C., with 250 rpm shaking for 10 min. The biotin antibody mixture comprises IgA+IgG+IgM (KPL, Cat#16-10-07). The target binding solution is removed and the plates are washed three times with 150 mM NaNO3/0.3% Tween. Next, free streptavidin (SA) (10 ng/μL) is allowed to bind by adding 100 μL to each well. The slides are incubated at 23° C., with 250 rpm shaking for 10 min. The SA solution is removed and the plates are washed three times with 150 mM NaNO3/0.3% TW. Next, 100 μL of Biotin-conjugated gold nanoparticle probe (0.214 μL biotin-Au probe/100 uL binding buffer) is added each well and the slides are incubated at 23° C. with shaking at 250 rpm for 10 min. The nanoparticle solution is removed and the plates are washed two times with 150 mM NaNO3/0.3% Tween.

Silver development is then used to enhance the images. Briefly, silver solutions A (Part # E700074D007) and B6 (Part # E700251D001) are mixed in a 50 mL of tube and added to a slide container. The slides are incubated at 120 rpm for 5.5 min at room temperature (23° C.). After silver development, the slides are rinsed with copious amounts of deionized water (at least 100 mL/slide). The slides are dried by spinning and the back of the slides are cleaned with a soft cloth or tissue. Finally, the slides are imaged with the Verigene System at 1.8 ms, 3.9 ms and multiple exposures 6× (10 ms, 20 ms, 50 ms, 100 ms, 200 ms, 500 ms, 1000 ms). Signal is the relative numerical signal response taken from the image of a scan from a Tecan LS scanner with data extraction and quantitation performed using GenePix software (Axon Instruments).

### Example 4

It is well known that amyloid and a variety of Tau forms exist in human CSF. Individually, they have been used as targets to develop diagnostic approach for Alzheimer's disease. However, none of those targets alone is definitive.

As described below, a complex of amyloid and Tau was present in serum. This complex can serve as a new target for development of a diagnostic assay for Alzheimer's disease.

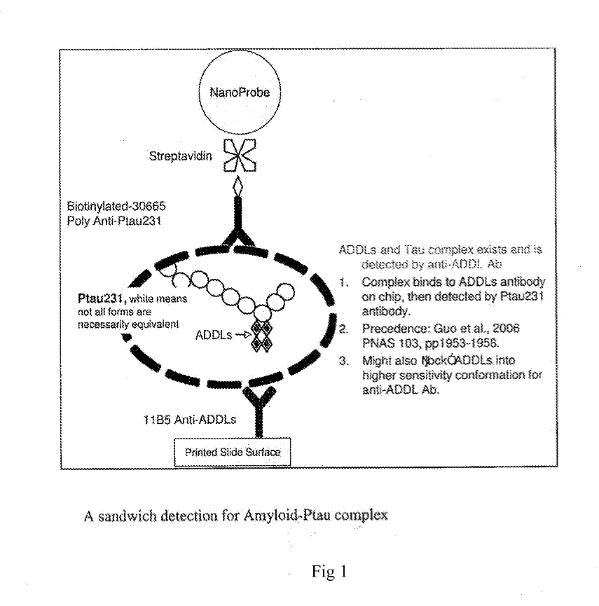

Two unique antibodies were used in the assay: amyloid oligomer specific antibody 11B5 (from Northwestern) was printed on chips and used as capturing antibody; Tau231 specific antibody Ab30665 was biotinylated and used for detecting. An Ab-target-Ab sandwich scheme is used for detection. When a specific target is present, which can bind to both antibodies simultaneously, a sandwich forms and generates detectable signal (FIG. 1). Note that the nanoprobes include biotin. Blinded serum samples were tested using the Nanosphere ultrasensitive protein detection platform. Within a total of 110 serum samples, 5 showed positive signals in this sandwich detection format.

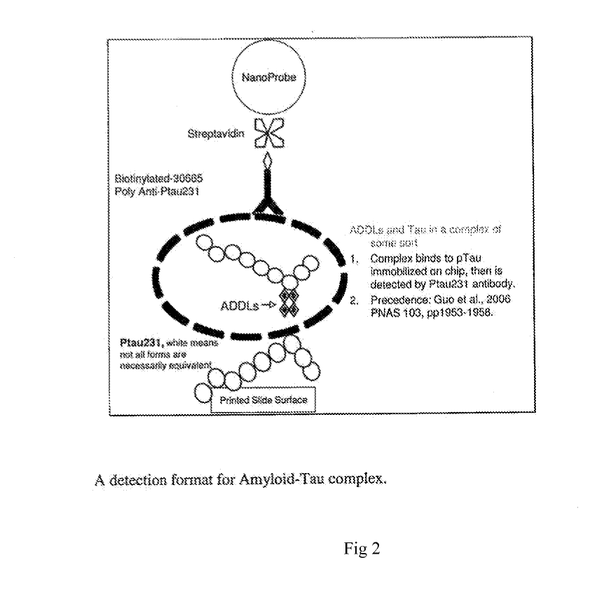

An alternative assay format is shown in FIG. 2. Phosphorylated Tau is printed on chips as a capture reagent, and anti-Tau 231 antibody is biotinylated and serves as a detection antibody. The presence of complexes is detected when a signal is generated that is greater than a control, e.g., the signal in wells without a serum sample.

### Example 5

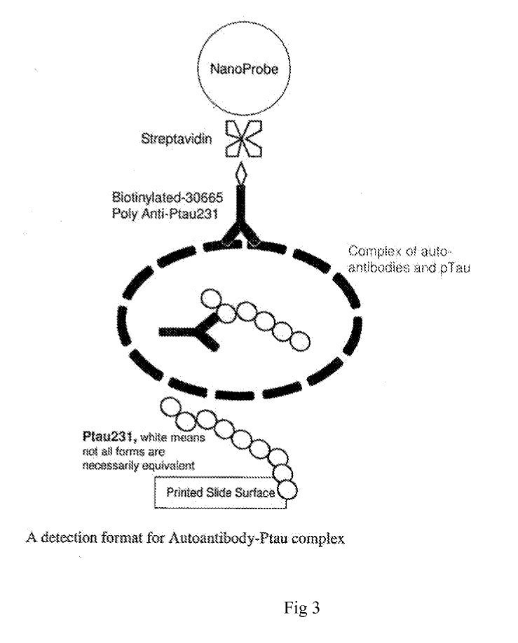

Phosphorylated Tau is printed on chips as a capture. Anti-Tau 231 antibody is biotinylated and serves as adetection antibody. If a Tau autoantibody-antigen complex is present in serum, it binds to Tau on the chip, forming a bridge between the capture reagent and the complex in serum, and is detected by anti-Tau antibody (FIG. 3). A signal is then generated and detected as described above. 5 samples among the 110 serum samples tested were positive in this assay.

### Example 6

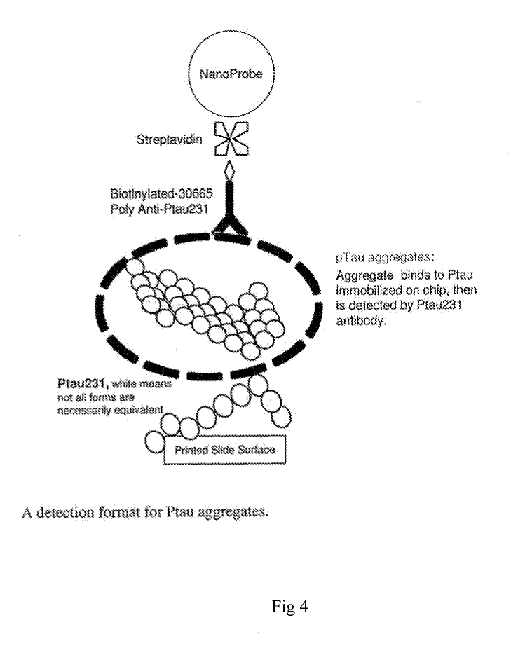

Phosphorylated Tau is printed on chips as capture. Anti-Tau 231 antibody is biotinylated and serves as detection antibody. Tau can aggregate into oligomers. This oligomer binds to the Tau printed on chip and then is detected by Tau antibody (FIG. 4).

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which can, of course, vary. It is also to be understood that the terminology used herein is for the purpose of describing particular embodiments only, and is not intended to be limiting.

As will be understood by one skilled in the art, for any and all purposes, particularly in terms of providing a written description, all ranges disclosed herein also encompass any and all possible subranges and combinations of subranges thereof.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

All publications, patent applications, patents, and other references mentioned herein are expressly incorporated by reference in their entirety, to the same extent as if each were incorporated by reference individually. In case of conflict, the present specification, including definitions, will control.

What is claimed is:

1. A method to diagnose Alzheimer's disease in a subject comprising:
(a) providing a substrate having a capture probe bound thereto, wherein the capture probe comprises i) an antibody specific for Tau, or aggregates thereof, Abeta, ADDLs, globulomers, variants thereof or fragments thereof, or ii) comprises an antigen comprising Tau, or aggregates thereof, Abeta, ADDLs, globulomers, variants thereof or fragments thereof;
(b) contacting the substrate having the capture probe bound thereto with (i) a physiological blood fluid sample from the subject and (ii) a detection probe under conditions that are suitable for the formation of a complex comprising the capture probe, the detection probe and the Tau, or aggregates thereof, Abeta, ADDLs, globulomers, variants thereof or fragments thereof, or complexes thereof, if present in the sample, wherein the detection probe comprises an antibody that specifically binds Tau, or aggregates thereof, Abeta, ADDLs, globulomers, variants thereof or fragments thereof, or complexes thereof; and
(c) detecting the formation of the complex having the capture probe and detection probe, wherein the presence of the complex having the capture probe and detection probe is indicative of complexes of Tau or aggregates thereof, and Abeta, ADDLs, or globulomers, or variants thereof or fragments thereof, in the subject.
